SEQUENCE LISTING (9 sequences)

SEQ ID NO: 1 - protein, 4776 aa, Streptococcus pneumoniae
MTETVEDKVSHSITGLDILKGIVAAGAVISGTVATQTKVFTNESAVLEKTVEKTDALATNDTVVLGTISTSNSASSTSLSASESASTSASESASTSASTSASTSASESASTSASTSISASSTVVGSQTAAATEATAKKVEEDRKKPASDYVASVTNVNLQSYAKRRKRSVDSIEQLLASIKNAAVFSGNTIVNGAPAINASLNIAKSETKVYTGEGVDSVYRVPIYYKLKVTNDGSKLTFTYTVTYVNPKTNDLGNISSMRPGYSIYNSGTSTQTMLTLGSDLGKPSGVKNYITDKNGRQVLSYNTSTMTTQGSGYTWGNGAQMNGFFAKKGYGLTSSWTVPITGTDTSFTFTPYAARTDRIGINYFNGGGKVVESSTTSQSLSQSKSLSVSASQSASASASTSASASASTSASASASTSASASASTSASVSASTSASASASTSASASASTSASESASTSASASASTSASASASTSASASASTSASESASTSASASASTSASESASTSASASASTSASASASTSASGSASTSTSASASTSASASASTSASASASISASESASTSASESASTSTSASASTSASESASTSASASASTSASASASTSASASASTSASASTSASESASTSASASASTSASASASTSASASASTSASASASTSASVSASTSASASASTSASASASTSASESASTSASASASTSASASASTSASASASTSASASASTSASASASTSASESASTSASASASTSASASASTSASASASTSASASASTSASASASISASESASTSASASASTSASASASTSASASASTSASESASTSASASASTSASASASTSASASASTSASASASTSASASASTSASASASTSASESASTSASASASTSASESASTSASASASTSASASASTSASASASTSASASASTSASASASTSASASASTSASASTSASESASTSASASASTSASASASTSASASASTSASESASTSASASASTSASASASTSASASASTSASASASTSASASASISASESASTSASASASTSASVSASTSASASASTSASESASTSASASASTSASESASTSASASASTSASASASISASESASTSASASASTSASASASTSASASASTSASESASTSTSASASTSASESASTSASASASTSASASASTSASASASTSASASASTSASASTSASESASTSASASASTSASASASTSASASASTSASASASTSASASASTSASASASTSASASASTSASASASTSASESASTSASASASTSASASASTSASASASTSASASASTSASVSASTSASESASTSASASASTSASASASTSASESASTSASASASTSASESASTSASASASTSASASASTSASASASTSASASASTSASASASTSASASASTSASASASTSASASASTSASASASTSASASASTSASASASTSASASASTSASASASISASESASTSASASASTSASASASTSASVSASTSASASASTSASASASISASESASTSASASASTSASASASTSASASASTSASASASISASESASTSASASASTSASASASTSASASASTSASASASTSASASASTSASASASTSASASASTSASASASTSASASASTSASESASTSASASASTSASASASTSASASASTSASVSASTSASESASTSASASASTSASASASTSASASASTSASESASTSASASASTSASASASTSASESASTSASASASTSASASASTSASASASTSASASASASTSASASASTSASASASTSASASASISASESASTSASESASTSTSASASTSASESASTSASASASTSASASASTSASASASTSASASTSASESASTSASASASTSASASASTSASASASTSASASASTSASASASTSASVSASTSASASASTSASASASTSASESASTSASASASTSASASASTSASASASTSASASASTSASASASTSASESASTSASASASTSASASASTSASASASTSASASASTSASASASISASESASTSASASASTSASASASTSASASASTSASESASTSASASASTSASASASTSASASASTSASASASTSASASASTSASASASTSASESASTSASASASTSASESASTSASASASTSASASASTSASASASTSASASASTSASASASTSASASASTSASASTSASESASTSASASASTSASASASTSASASASTSASESASTSASASASTSASASASTSASASASTSASASASTSASASASISASESASTSASASASTSASVSASTSASASASTSASESASTSASASASTSASESASTSASASASTSASASASISASESASTSASASASTSASASASTSASASASTSASESASTSTSASASTSASESASTSASASASTSASASASTSASASASTSASASASTSASASTSASESASTSASASASTSASASASTSASASASTSASASASTSASASASTSASASASTSASASASTSASASASTSASASASTSASASASTSASASASTSASESASTSASASASTSASASASTSASASASTSASVSASTSASESASTSASASASTSASASASTSASASASTSASESASTSASASASTSASASASTSASESASTSASASASTSASASASTSASASASTSASASASASTSASASASTSASASASTSASASASISASESASTSASASASASTSASASASTSASASASTSASASASISASESASTSASESASTSTSASASTSASESASTSASASASTSASASASTSASASASTSASASTSASESASTSASASASTSASASASTSASASASTSASASASTSASASASTSASVSASTSASASASTSASASASTSASESASTSASASTSASESASTSASASASTSASASASTSASASASTSASESASTSASASASTSASASASTSASESASTSASASASTSASASASTSASASASTSASESASTSASASASTSASESASTSASASASTSASASASTSASGSASTSTSASASTSASASASTSASASASISASESASTSASESASTSTSASASTSASESASTSASASASTSASASASTSASASASTSASASTSASESASTSASASASTSASASASTSASASASTSASASASTSASVSASTSASASASTSASASASTSASESASTSASASASTSASASASTSASASASTSASASASTSASASASTSASESASTSASASASTSASASASTSASASASTSASASASTSASASASISASESASTSASASASTSASASASTSASASASTSASESASTSASASASTSASASASTSASASASTSASASASTSASASASTSASASASTSASESASTSASASASTSASESASTSASASASTSASASASTSASASASTSASASASTSASASASTSASASASTSASASTSASESASTSASASASTSASASASTSASASASTSASESASTSASASASTSASASASTSASASASTSASASASTSASASASISASESASTSASASASTSASVSASTSASASASTSASESASTSASASASTSASESASTSASASASTSASASASISASESASTSASASASTSASASASTSASASASTSASESASTSTSASASTSASESASTSASASASTSASASASTSASASASTSASASASTSASASTSASESASTSASASASTSASASASTSASASASTSASASASTSASASASTSASASASTSASASASTSASASASTSASESASTSASASASTSASASASTSASASASTSASASASTSASVSASTSASESASTSASASASTSASASASTSASESASTSASASASTSASESASTSASASASTSASASASTSASASASTSASASASTSASASASTSASASASTSASASASTSASASASTSASASASTSASASASTSASASASTSASASASTSASASASISASESASTSASASASTSASASASTSASVSASTSASASASTSASASASISASESASTSASASASTSASASASTSASASASTSASASASISASESASTSASASASTSASASASTSASASASTSASASASTSASASASTSASASASTSASASASTSASASASTSASASASTSASESASTSASASASTSASASASISASESASTSASASASTSASASASTSASASASTSASESASTSTSASASTSASESASTSASASASTSASASASTSASASASTSASASASTSASASTSASESASTSASASASTSASASASTSASASASTSASASASTSASASASTSASASASTSASASASTSASASASTSASESASTSASASASTSASASASTSASASASTSASASASTSASVSASTSASESASTSASASASTSASASASTSASESASTSASASASTSASESASTSASASASTSASASASTSASASASTSASASASTSASASASTSASASASTSASASASTSASASASTSASASASTSASASASTSASASASTSASASASTSASASASISASESASTSASASASTSASASASTSASVSASTSASASASTSASASASISASESASTSASASASTSASASASTSASASASTSASASASISASESASTSASASASTSASASASTSASASASTSASASASTSASASASTSASASASTSASASASTSASASASTSASASASTSASASASTSASASASTSASASASTSASASASTSASASASTSVSNSANHSNSQVGNTSGSTGKSQKELPNTGTESSIGSVLLGVLAAVTGIGLVAKRRKRDEEE

SEQ ID NO: 2 - DNA, 31 bp, Artificial
  Artificial primer
nnnnnactagtggagataatcagtctgcttg

SEQ ID NO: 3 - DNA, 29 bp, Artificial
  Artificial primer
nnnnnaagcttgcgcgctcataagtcgcc

SEQ ID NO: 4 - DNA, 31 bp, Artificial
  Artificial primer
nnnnntctagaggcaagtacatctgcatctg

SEQ ID NO: 5 - DNA, 31 bp, Artificial
  Artificial primer
nnnnngcggccgcgatagaatatccaggacg

SEQ ID NO: 6 - DNA, 32 bp, Artificial
  Artificial primer
nnnnngaattctcagcgagttcaactagtttg

SEQ ID NO: 7 - DNA, 28 bp, Artificial
  Artificial primer
nnnnnaagcttttaactagcacttactg

SEQ ID NO: 8 - DNA, 25 bp, Artificial
  Artificial primer
nnnnngaattcatgaaaaaacgagc

SEQ ID NO: 9 - DNA, 30 bp, Artificial
  Artificial primer
nnnnnaagcttacttcctgaaaataggagc

(19) United States
(12) Patent Application Publication
(10) Pub. No.: US 2011/0171224 A1
(43) Pub. Date: Jul. 14, 2011

(54) PsrP IS A PROTECTIVE ANTIGEN AGAINST PNEUMOCOCCAL INFECTION

(76) Inventor: Carlos J. Orihuela, San Antonio, TX (US)

(21) Appl. No.: 12/994,081

(22) PCT Filed: May 22, 2009
(86) PCT No.: PCT/US09/45071

§ 371 Date: Jan. 27, 2011

Related U.S. Application Data
(60) Provisional application No. 61/055,378, filed on May 22, 2008.

(51) Int. Cl. A61K 39/395; A61K 39/09; A61K 31/7088; A61P 37/04; A61P 31/04

(52) U.S. Cl. 424/139.1; 424/244.1; 514/44 R; 424/165.1; 424/190.1

(57) ABSTRACT

Disclosed are pharmaceutical compositions that include a PsrP polypeptide, a nucleic acid encoding a PsrP polypeptide, or an antibody or fragment thereof that binds to a PsrP polypeptide domain, and methods of inhibiting, modulating, treating, or preventing a bacterial infection, such as an infection due to Streptococcus pneumoniae, in a subject using these compositions.

The present application claims the benefit of priority to U.S. Provisional Patent Application No. 61/055,378, filed May 22, 2008, the entire contents of which is hereby incorporated by reference in its entirety.

### BACKGROUND OF THE INVENTION

1. Field of the Invention

The present invention relates generally to the fields of immunology, microbiology, and infectious diseases. More particularly, it concerns compositions that include a PsrP polypeptide, a nucleic acid encoding a PsrP polypeptide, or an antibody or fragment thereof that binds to a PsrP polypeptide domain, and methods of inhibiting, modulating, treating, or preventing a bacterial infection in a subject using these compositions.

2. Description of Related Art

Streptococcus pneumoniae (the pneumococcus) is a leading cause of community-acquired pneumonia, sepsis, and meningitis. Primarily a commensal bacterium, invasive pneumococcal disease (IPD) is characterized by spread of the pneumococcus from the nasopharynx to normally sterile sites such as the lungs, blood, and brain. At risk for IPD are young children, the elderly, and individuals who are immunocompromised. Worldwide, it is estimated that S. pneumoniae is responsible for 15 cases of IPD per 100,000 persons per year and over a million deaths annually (WHO Position Paper, 1999; ACIP (2000). Of note, the preponderance of IPD is the result of infection with relatively few invasive clones (Sandgren et al., 2004), a finding that suggests invasive clones carry genes that facilitate disease 66 progression and are absent in non-invasive isolates.

In 2006, it was determined that the presence of psrP-secY2A2, a 37-kb 68 pathogenicity island, was positively correlated with the ability of S. pneumoniae to cause invasive disease (Obert et al., 2006). Analysis of operon structure and gene content found psrP-secY2A2 to be highly homologous to loci in oral streptococci and some isolates of Staphylococcus aureus that encode a family of glycosylated proteins the inventors now call serine-rich repeat proteins (SRRPs) (Bensing and Sullam, 2002; Froeliger and Fives-Taylor, 2001; Handley et al., 2005; Siboo et al., 2005; Takahashi et al., 2002). SRRPs are adhesins and have been implicated in biofilm formation, colonization of the dental surface, and the development of infective endocarditis (Froeliger and Fives-Taylor, 2001; Bensing et al., 2004a; Bensing et al., 2004b; Chen et al., 2004; Takamatsu et al., 2005; Wu et al., 1998). For example, Fap1, the Streptococcus sanguis SRRP, is required for biofilm formation on glass (Froeliger and Fives-Taylor, 2001). Likewise, GspB and SraP, the Streptococcus gordonii and S. aureus SRRPs, respectively, contribute towards formation of vegetative plaques on heart valves of catheterized rats (Siboo et al., 2005; Bensing et al., 2004a; Takahashi et al., 2006). Thus SRRPs serve to anchor bacteria to host surfaces.

S. pneumoniae serotype 4, strain TIGR4 contains psrP-secY2A2 which encodes the SRRP Pneumococcal serine-rich repeat protein (PsrP) (Tettelin et al., 2001). PsrP is composed of 4776 amino acids and is the longest bacterial protein known (Obert et al., 2006). Like other SRRPs, PsrP consists of a large cleavable signal peptide, a short serine-rich repeat region (SRR1), a basic region (BR) followed by a second extremely long serine rich repeat area (SRR2), and a cell wall anchor domain located at the carboxy terminus (FIG. 1). Briefly, the SRR1 and SRR2 domain of PsrP are composed of 8 and 539 repeats of the amino acid sequence SAS[A/E/V]SAS[T/I], respectively. psrP-secY2A2 also encodes 10 glycosylases and an alternate SecY2A2 translocase composed of 7 proteins. Based on their near-identical homology to genes within the S. gordonii GspB locus gspB-secY2A2 (Obert et al., 2006), these genes are putatively responsible for the glycosylation and transport of PsrP (Bensing and Sullam, 2002; Bensing et al., 2004; Bensing et al., 2004b; Takamatsu et al., 2005; Takamatsu and Bensing, 2006; Bensing et al., 2005; Takamatsu et al., 2005; Takamatsu et al., 2004; Takamatsu et al., 2004). PsrP is the first SRRP to be linked to respiratory tract disease and its disruption has been shown to attenuate TIGR4 virulence in mice (Obert et al., 2006).

Thus, given the high prevalence and morbidity associated with bacterial infections, there is the need for more effective treatment or preventive measures.

### SUMMARY OF THE INVENTION

The present invention is in part based on the finding that PsrP is a protective antigen against bacterial infection. For example, it has been found that PsrP antigen protects mice against challenge with the respiratory tract pathogen Streptococcus pneumoniae. Thus, strategies employing PsrP or targeting PsrP have application in the treatment and prevention of bacterial infections.

The present invention generally concerns pharmaceutical compositions that include (1) a PsrP polypeptide or a nucleic acid encoding a PsrP polypeptide, and (2) a pharmaceutically acceptable carrier. The composition may induce a humoral or cell-mediated immune response when administered to a subject. The compositions of the present invention can be applied in the treatment and prevention of bacterial disease.

The present invention is also generally directed to methods of inhibiting, modulating, treating, or preventing a bacterial infection or the symptoms thereof in a subject, comprising administering an effective amount of a pharmaceutical composition comprising a PsrP polypeptide and a pharmaceutically acceptable carrier, wherein the bacterial infection is inhibited, modulated, treated, or prevented. The bacterial infection may be any type of bacterial infection. In particular embodiments, the bacterial infection is due to Streptococcus pneumoniae. The infection may be an infection of any part of the subject, but in specific embodiments the infection causes pneumonia.

“Subject” as used herein may mean fish, amphibians, reptiles, birds, and mammals, such as mice, rats, rabbits, goats, cats, dogs, cows, apes and humans.

In some embodiments, the PsrP polypeptide comprises 50 to 200 consecutive amino acids of SEQ ID NO:1 (which is Genbank Accession Number AAK75846). In more particular embodiments, the polypeptide comprises 50 to 500 consecutive amino acids of SEQ ID NO:1. In even more particular embodiments, the PsrP polypeptide comprises 50 to 1000 consecutive amino acids of SEQ ID NO:1. In other embodiments, the PsrP polypeptide comprises 50 to 2000 consecutive amino acids of SEQ ID NO:1. In more specific embodiments, the pharmaceutical composition comprises 50 to 4000 consecutive amino acids of SEQ ID NO:1. In a particular embodiment, the PsrP polypeptide comprises SEQ ID NO:1.

In further embodiments, the PsrP polypeptide comprises a region that has at least 70% sequence identity to a consecutive series of at least 100 amino acids of SEQ ID NO:1, at least 80% sequence identity to a consecutive series of at least 100 amino acids of SEQ ID NO:1, at least 90% sequence identity to a consecutive series of at least 100 amino acids of SEQ ID NO:1, at least 95% sequence identity to a consecutive series of at least 100 amino acids of SEQ ID NO:1, or at least 99% sequence identity to a consecutive series of at least 100 amino acids of SEQ ID NO:1.

In some embodiments, the PsrP polypeptide comprises a serine-rich polypeptide derived from SEQ ID NO:1. For example, the PsrP polypeptide may comprises at least 100 consecutive amino acids of SEQ ID NO:1, wherein at least about 30% of the amino acid residues in the at least 100 consecutive amino acids of SEQ ID NO:1 are serine residues. In further embodiments, at least 40%, 50%, 60%, or 70% of the amino acid residues are serine residues. Other examples of PsrP polypeptides are discussed in the specification below.

The PsrP polypeptide may or may not be comprised in an antigen-presenting cell. In such a cell, the PsrP polypeptide would be considered an antigen. For example, the pharmaceutical composition may include dendritic cells expressing one or more PsrP polypeptides on their cell surface.

The pharmaceutical composition may include one or more additional components. For example, the composition may include one or more adjuvants. In other embodiments, one or more of the additional agent(s) is covalently bonded to the PsrP polypeptide. In some embodiments, one or more vaccine components may be comprised in a lipid or liposome. A composition of the present invention, and its various components, may be prepared and/or administered by any method disclosed herein or as would be known to one of ordinary skill in the art, in light of the present disclosure.

The present invention is also directed to pharmaceutical compositions that include an antibody or fragment thereof that binds to a PsrP antigen and a pharmaceutically acceptable carrier. In particular embodiments, the antibody or fragment thereof binds to a domain within SEQ ID NO:1. In more particular embodiments, the antibody or fragment binds to the basic domain of PsrP or the serine-rich repeat region of PsrP.

The composition may be administered using any method known to those of ordinary skill in the art. Non-limiting examples include administration by aerosol, by spray, intravenously, intradermally, intraarterially, intramuscularly, intrathecally, intratracheally, subcutaneously, orally, topically, or intraperitoneally.

In some embodiments, dendritic cells are obtained from either the subject to be treated or a donor subject, and are transduced ex vivo to express a PsrP polypeptide on the cell surface. In other embodiments, dendritic cells are transduced in vivo in a subject. The transduced dendritic cells, if transduced ex vivo, would then be administered to the subject.

In some embodiments, the composition comprises a pharmaceutical composition comprises a nucleic acid encoding a PsrP polypeptide or encoding an antibody or fragment of an antibody that binds to a PsrP polypeptide. The nucleic acid may be comprised in an expression cassette that includes a promoter that is operatively coupled to the nucleic acid. In some embodiments, the nucleic acid is comprised in a vector. For example, the vector may be a cell. Non-limiting examples of cells include antigen-presenting cells, and white blood cells. For example, the antigen-presenting cell may be a dendritic cell or a macrophage. The expression cassette may optionally be delivered via a viral vector. Non-limiting examples of viral vectors include adenovirus, adeno-associated virus, retrovirus, and lentivirus. In some embodiments, the vector is a liposome. Any vector known to those of ordinary skill in the art is contemplated by the present methods, some of which are discussed in further detail in the specification below.

The PsrP polypeptide may be any of those PsrP polypeptides discussed above. In particular embodiments the antibody or fragment thereof binds to the basic domain of PsrP or the serine-rich repeat region of PsrP.

The invention also generally concerns methods of inhibiting, modulating, treating, or preventing a bacterial infection or the symptoms thereof in a subject, that involve administering an effective amount of a pharmaceutical composition comprising an antibody or fragment thereof that binds to a PsrP polypeptide and a pharmaceutically acceptable carrier to the subject, wherein the bacterial infection is inhibited, modulated, treated, or prevented. The subject may be any of those subjects discussed above. In particular embodiments, the subject is a human subject. The bacterial infection may be any type of bacterial infection, but in specific embodiments the bacterial infection is due to Streptococcus pneumoniae. The infection may be an infection of any part of the subject, but in specific embodiments the infection causes pneumonia. In particular embodiments, the antibody or fragment thereof binds to a domain within SEQ ID NO:1. In specific embodiments, the antibody or fragment binds to the basic domain of PsrP or the serine-rich repeat region of PsrP. In some embodiments, the antibody or fragment thereof is administered by administering a nucleic acid encoding an antibody or fragment thereof.

The present invention also concerns kits that include a sealed vial that includes a pharmaceutical composition which includes: (a) a PsrP polypeptide, a nucleic acid encoding a PsrP polypeptide, an antibody that binds to a PsrP polypeptide, or an antibody fragment that binds to a PsrP polypeptide; and (b) a pharmaceutically acceptable carrier. The kit may include one or more additional vials. In some embodiments, the kit further includes a syringe, or instructions for use. The pharmaceutically acceptable carrier may be any pharmaceutically acceptable carrier known to those of ordinary skill in the art. In particular embodiments, the pharmaceutically acceptable carrier is an aqueous carrier.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device and/or method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention. The invention may be better understood by reference to one or more of these drawings in combination with the detailed description of specific embodiments presented herein.

FIG. 1. Schematic representation of PsrP. PsrP is composed of a unique signal peptide (SP), a short serine-rich repeat region (SRR1), a basic region (BR), an extremely long second serine-rich repeat region (SRR2), and a cell wall anchor domain (CWAD). SRR1 and SRR2 are composed of 8 and 539 repeats of the amino acid sequence SAS[A/E/V]SAST/I, respectively. PsrP is the longest bacterial protein known.

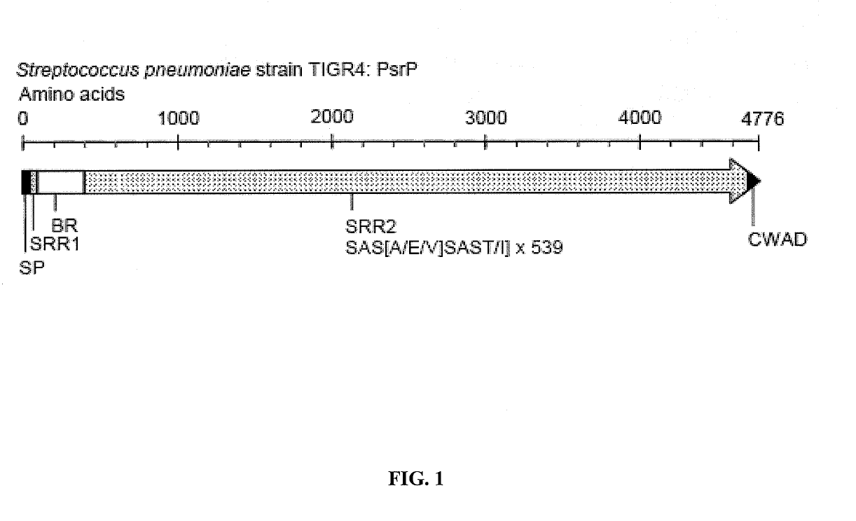

FIG. 2 T4ΔpsrP is an isogenic mutant. A) Northern blots examining transcription of psrP (SP1772) and the genes located immediately upstream (SP1773) and downstream (glyA) of psrP in TIGR4 (T4), the mutantsT4ΔpsrP (Ä) and T4ΔpsrP-secY2A2 (Ω), and D39, a serotype 2 strain that does not carry psrP-secy2A2; note that SP1773 and glyA expression were unaffected in the mutant T4ΔpsrP. This suggests that psrP has its own promoter and that T4ΔpsrP is an isogenic mutant. Note also that SP1773 expression is unaffected in the mutant T4ΔpsrP-secY2A2, whereas psrP and glyA expression are abrogated. This is because T4ΔpsrP-secY2A2 was created by allelic exchange and lacks SP1772-SP1757. Normal expression of SP1773 in T4ΔpsrP-secY2A2 indicates that there were no upstream effects of the allelic exchange. B) An ethidium bromide stained gel demonstrating that equivalent amounts of RNA were used to examine gene transcription.

FIG. 3 PsrP is required in the lungs but not the nasopharynx or blood. Bacterial titers of individual 5 week old female Balb/cJ mice infected with TIGR4, T4ΔpsrP, and T4ΔpsrP-secY2A2 in nasopharyngeal lavage, lungs, and blood. Mice were infected via intranasal (10⁷ cfu in 20 μl), intratracheal (10⁵ cfu in 100 μl), and intraperitoneal (10⁵ cfu in 100 μl) challenge routes. Samples were collected at 48, 48, and 24 hours post-challenge, respectively. Horizontal bars indicate median value. Statistical analysis was performed using a 1-Way ANOVA. Asterisks indicate a statistically significant difference versus wild type values.

FIG. 4 PsrP contributes towards adhesion to lung cells but not pharyngeal or capillary endothelial cells. In vitro adhesion assays measuring the ability of TIGR4, T4ΔpsrP and T4ΔpsrP-secY2A2 to adhere to human and rodent cell lines. Human cells included: a pharyngeal epithelial cell line (Detroit), type II pneumocytes (A549), and brain microvasculature endothelial cells (HBMEC). Rodent cell lines used were: mouse bronchial epithelial cells (LA-4), and rat brain capillary endothelial cells (RBCEC₆). Statistical analysis was performed using 1-Way ANOVA. Asterisks indicate a statistically significant difference versus wild type values.

FIG. 5 The amino terminus of PsrP contributes towards attachment to A549 cells. A) SDS-PAGE of purified rPsrP_{SRR1-BR}. The predicted size of the protein is 45 kDa. B-1) Attachment of latex microspheres coated with rPsrP_{SRR1-BR} (rPsrP) or bovine serum albumin (BSA) to A549 cells in vitro per field of vision (FOV) at 200×. B-2) Images of latex coated beads attached to A549 cells in vitro (400×). C) Dose-dependent competitive inhibition of TIGR4 binding to A549 cells following 30-minute incubation with rPsrP_{SRR1-BR}. Statistical analysis was performed using a 1-Way ANOVA.

FIG. 6 Antibodies against rPsrP_{SRR1-BR} inhibit S. pneumoniae adhesion in a dose-dependent manner. TIGR4 was incubated for 30 minutes with rabbit antiserum against rPsrP_{SRR1-BR}, naïve serum from the same animal, and antiserum against rSP0925, an unrelated S. pneumoniae protein, and tested for adherence to A549 cells. Statistical analysis was performed using a 1-Way ANOVA.

FIG. 7 Passive immunization of mice with rabbit antiserum against rPsrP_{SRR1-BR} protects mice against pneumococcal challenge. Bacterial titers of individual 5 week old female Balb/cJ mice infected intranasally with TIGR4 (10⁷ cfu) following passive immunization with 250 μL of naïve or high-titer antiserum against rPsrP_{SRR1-BR} and antiserum against rSP0925. Antiserum was delivered by intraperitoneal injection, one day prior to challenge. Horizontal bars indicate median value. Statistical analysis was performed using a 1-Way ANOVA.

FIG. 8 Model of PsrP on the S. pneumoniae surface. A) Cartoon illustration of PsrP domains. Note that SRR1 and SRR2 are glycosylated. B) PsrP is attached to peptidoglycan on the surface of the bacteria via the LXPTGE motif found in the cell wall anchor domain (CWAD). SRR2 serves as a stalk that allows the protein to protrude through the capsular polysaccharide and mediate attachment through the basic region (BR).

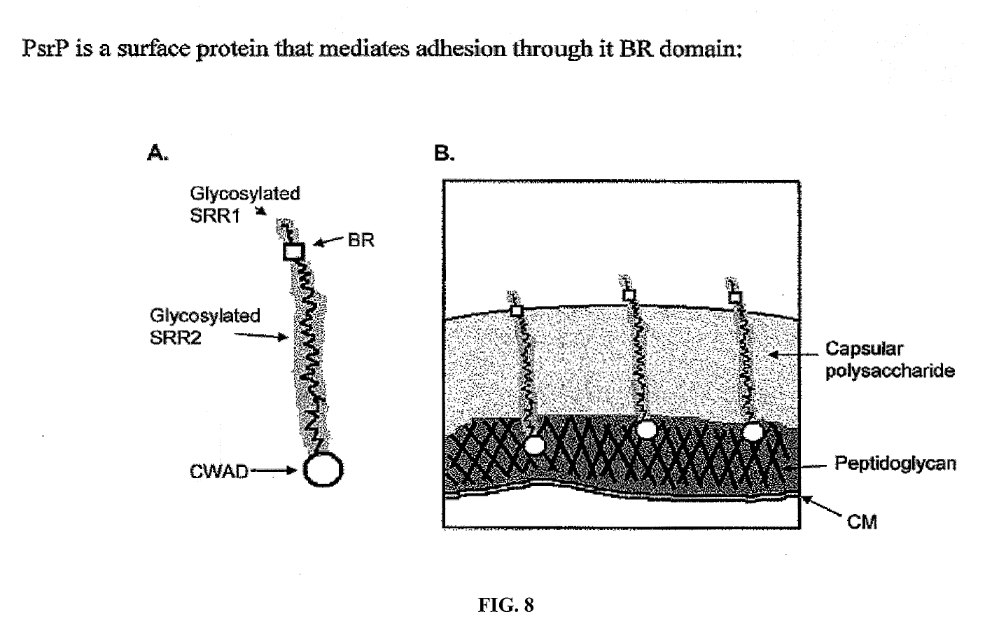

FIG. 9 The Basic Region (BR) domain of PsrP mediates adhesion and competitively inhibits attachment of S. pneumoniae to A549 cells. A) Attachment of latex microspheres coated with recombinant BR of PsrP (rPsrP_{BR}) or bovine serum albumin (BSA) to A549 cells in vitro. Field of vision (FOV) at 200×. B) Images of latex coated beads attached to A549 cells in vitro (FOV at 400×). C) Dose-dependent competitive inhibition of TIGR4 binding to A549 cells following 30-minute incubation with rPsrP_{BR}. Experiments were performed 3 times with statistical analysis performed using a One-Way ANOVA.

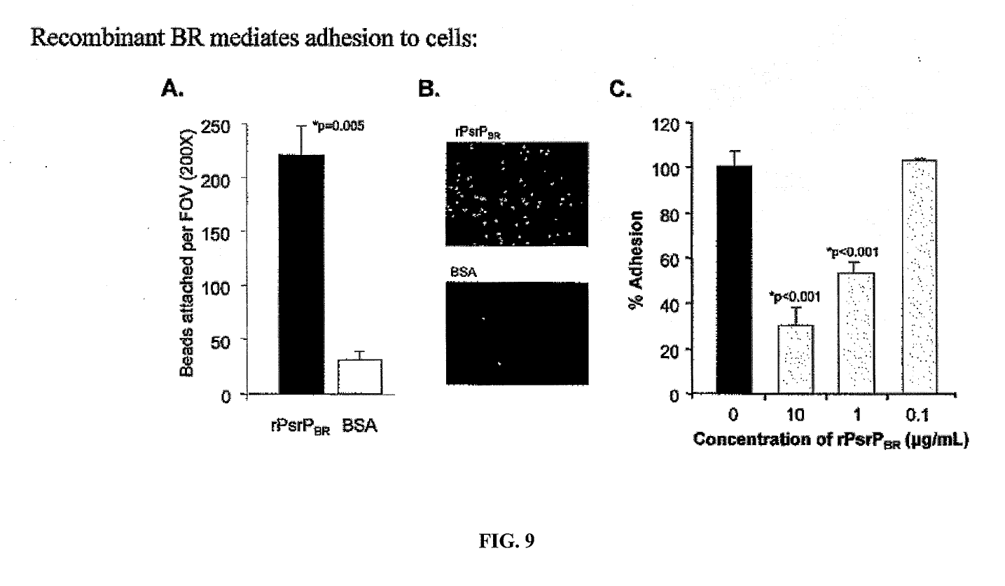

FIG. 10 Antiserum against rPsrP_{BR} protects against S. pneumoniae challenge. A) Incubation of TIGR4 for 30 minutes with rabbit antiserum against rPsrP_{BR} inhibited adhesion to A549 cells in a dose dependent manner (dilution 1:100 to 1:10,000). In contrast, bacteria incubated with naïve serum from the same rabbit adhered to cells ormall (i.e. no serum). Asterisks indicate statistical significance (p<0.05 as determined by a One-Way ANOVA. B) Passive immunization of mice with rabbit antiserum against rPsrP_{BR} protected mice against pneumococcal challenge. Median bacterial titers in relevant organs of mice (n=6) per cohort) passively immunized with 250 μl of naïve or high-titer antiserum against PsrP_{BR} serum and challenged with 10⁷ cfu of TIGR4. Antiserum was delivered by i.p. injection mice that received rPsrP_{BR} antiserum had ˜100-fold less bacteria in the lungs and blood than mice that received naïve serum. Statistical analysis was performed using a Mann-Whitney rank Sum Test.

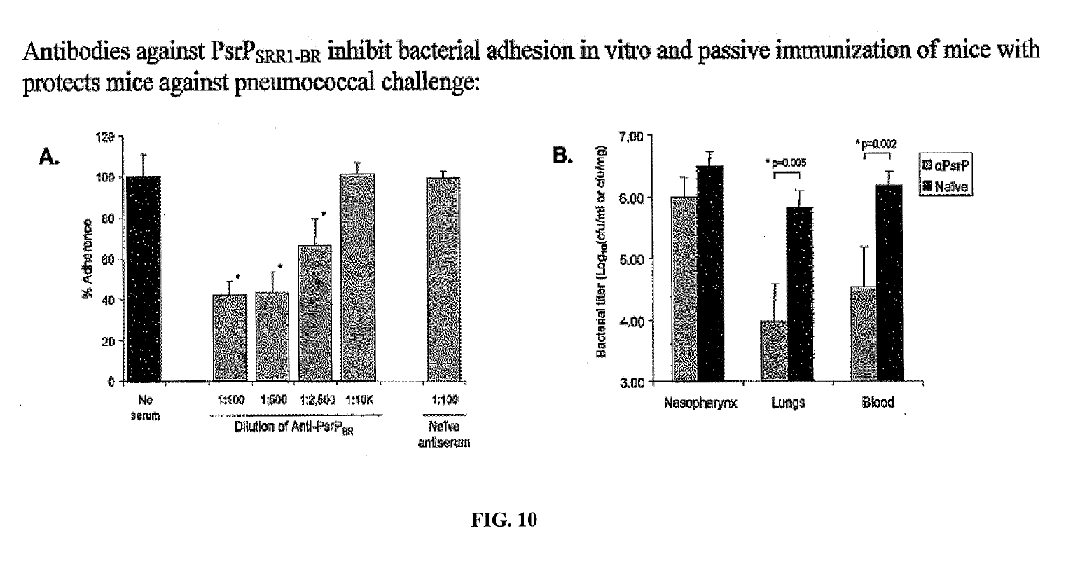

FIG. 11 Competitive inhibition of S. pneumoniae adhesion by PsrP BR fragments containing amino acids 273-341. A549 lung epithelial cells were incubated for 1 hour with tissue culture media containing 1 μM of recombinant PsrP SRR1-BR fragments or bovine serum albumin (BSA) as the negative control. Cells were washed and S. pneumoniae strain TIGR4 (107 cfu/mL) was added to the cells for 1 hour at 37° C. Adhered bacteria were quantitated by washing the cells and serial dilution of the cell lysate. Percent adherence values were normalized against those for BSA. Experiments were performed in triplicate and statistical analysis was performed using a 1-Way ANOVA. Asterisks indicate a p value ≦0.05. Competitive inhibition assays using consecutively smaller fragments of PsrP BR (Round 1-3) indicates that fragments containing AA 273-341 inhibit bacterial adhesion in a competitive manner. This suggests the binding domain of PsrP is located within this area.

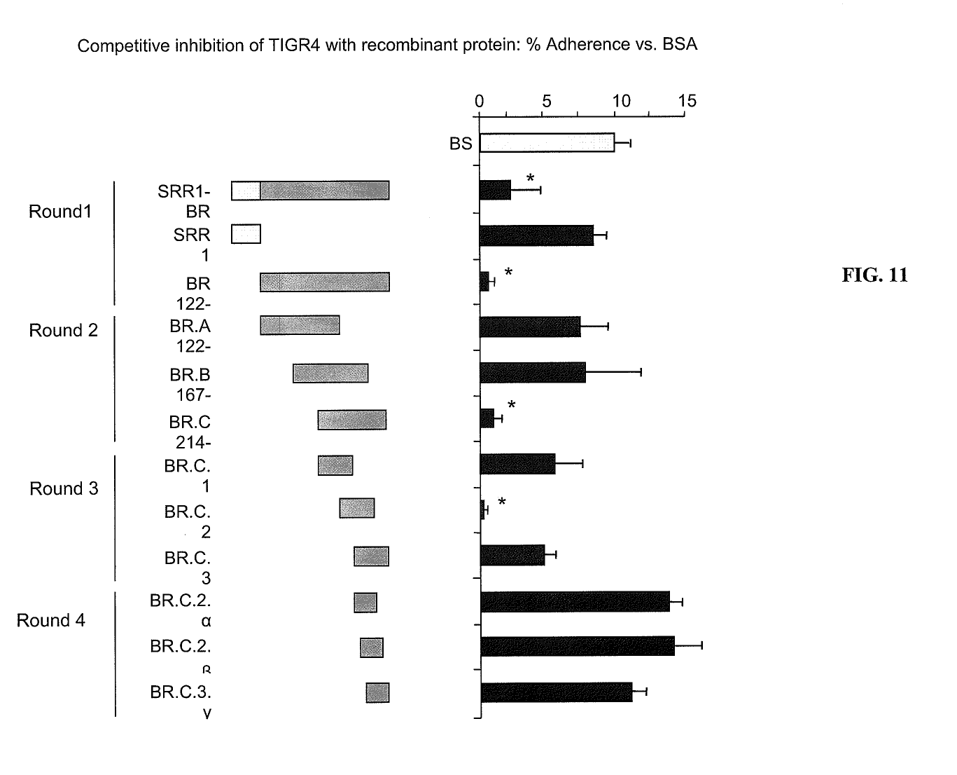

FIG. 12 Adhesion of latex beads coated with PsrP BR fragments containing amino acids 291-325. Latex microspheres were coated with recombinant PsrP SRR1-BR fragments or BSA as the negative control. Beads were added to confluent monolayer of A549 cells and incubated for 30 minutes at room temperature. Cells were washed with media to remove un unattached beads, beads adhered to the cells were visualized with an inverted fluorescent microscope. Experiments were performed in triplicate and statistical analysis was performed using a 1-Way ANOVA. Asterisks indicate a p value ≦0.05. Adhesion assays using consecutively smaller fragments of PsrP BR (Round 1-3) indicates that fragments containing AA 291-325 were able to adhere to cells. This suggests the binding domain of PsrP is located within this area.

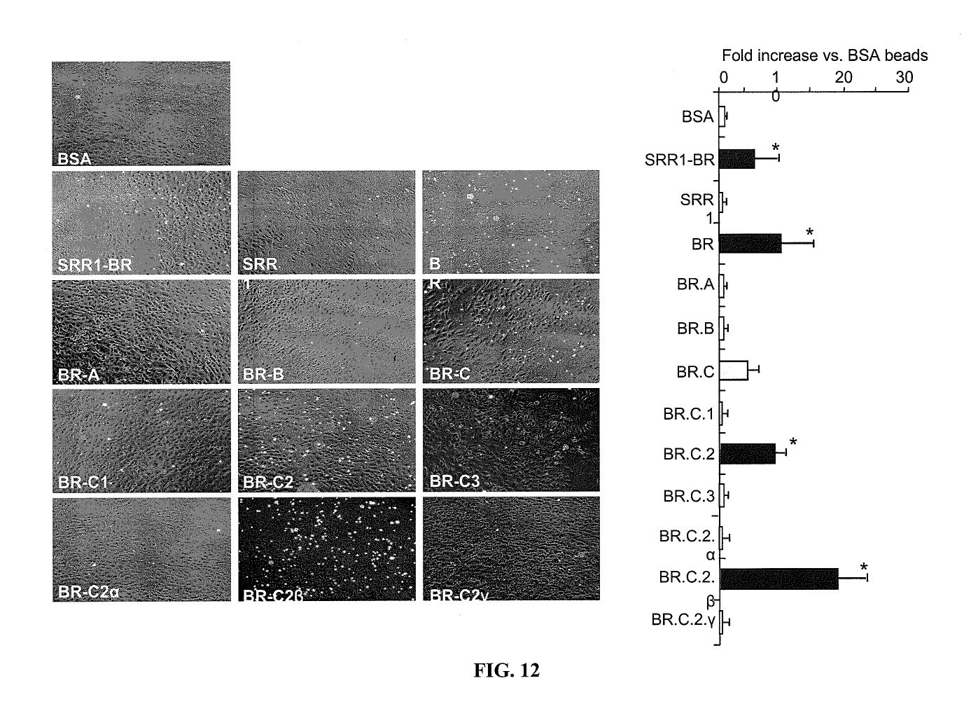

FIG. 13 PsrP fragments containing amino acid 273-341 bind to cells. Cy5 labeled PsrP SRR1-BR fragments were incubated with A549 cells, washed with media, then visualized with a fluorescent microscope. Proteins containing amino acids 273-341, adhered to cells. Note that we have not yet tested AA 291-325. This data shows that PsrP 273-341 binds to A549 cells.

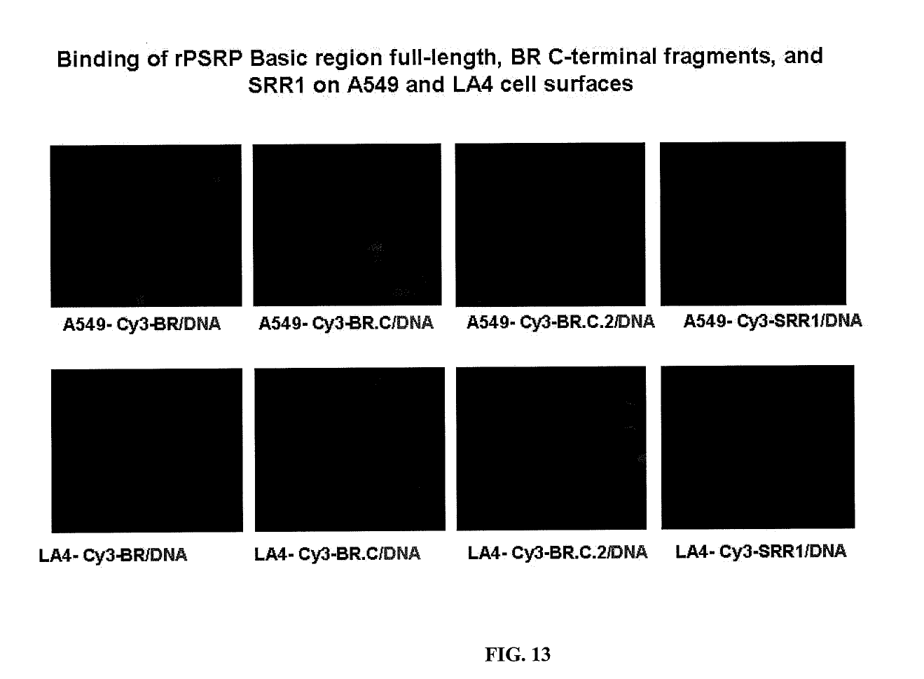

FIG. 14 Vaccination with SRR1-BR protects mice against pneumococcal challenge. Mice were vaccinated by intraperitoneal injection of recombinant PsrP SRR1-BR with Freund's complete adjuvant, and boosted twice with protein and incomplete adjuvant at 2 week intervals. Subsequently, mice were challenged with 107 cfu of S. pneumoniae strain TIGR4 intranasally. Panel A demonstrates decreased bacterial titers in the blood of mice vaccinated with SRR1-BR. This data shows that active vaccination of mice is protective against pneumococcal infection.

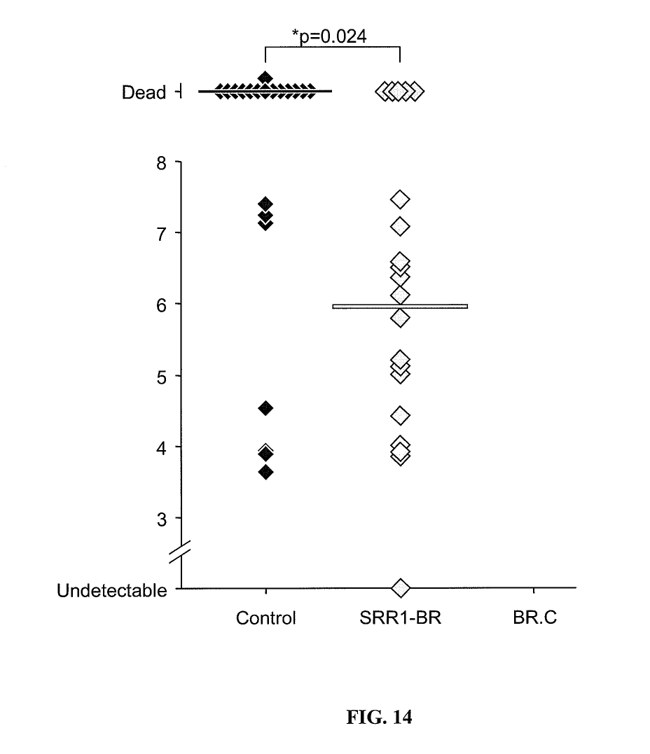

### DESCRIPTION OF ILLUSTRATIVE EMBODIMENTS

The present invention is based on the finding that PsrP is a protective antigen against bacterial infection. For example, it has been found that PsrP antigen protects mice against challenge with the respiratory tract pathogen Streptococcus pneumoniae.

### A. PsrP

1. PsrP Polypeptides

The present invention pertains to use of PsrP polypeptides in various contexts.

The full-length amino acid sequence of PsrP is provided herein, and is designated SEQ ID NO:1.

In the context of the present invention, a PsrP polypeptide is a consecutive amino acid segment of two or more amino acids of a PsrP. The PsrP can be a human PsrP or a PsrP from another mammal. In particular embodiments, the PsrP polypeptide is a polypeptide that includes two or more consecutive amino acids of SEQ ID NO:1. For example, the polypeptide may include 4, 5, 10, 15, 20, 25, 30, 35, 40, 45, 50, 55, 60, 65, 70, 75, 80, 85, 90, 95, 100, 120, 140, 160, 180, 200, 220, 240, 260, 280, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, or any number of consecutive amino acids of SEQ ID NO:1, or any range of amino acids derivable therein. For example, the PsrP polypeptide may comprise a consecutive series of 10 or more amino acids of SEQ NO:1, a consecutive series of 20 or more amino acids of SEQ ID NO:1, a consecutive series of 30 or more amino acids of SEQ ID NO:1, a consecutive series of 40 or more amino acids of SEQ ID NO:1, a consecutive series of 50 or more amino acids of SEQ ID NO:1, a consecutive series of 60 or more amino acids of SEQ ID NO:1, a consecutive series of 70 or more amino acids of SEQ ID NO:1, a consecutive series of 80 or more amino acids of SEQ ID NO:1, a consecutive series of 90 or more amino acids of SEQ ID NO:1, a consecutive series of 100 or more amino acids of SEQ ID NO:1, a consecutive series of 150 or more amino acids of SEQ ID NO:1, a consecutive series of 200 or more amino acids of SEQ ID NO:1, a consecutive series of 300 more or more amino acids of SEQ ID NO:1, a consecutive series of 400 or more amino acids of SEQ ID NO:1, a consecutive series of 500 or more amino acids of SEQ ID NO:1, a consecutive series of 600 or more amino acids of SEQ ID NO:1, a consecutive series of 700 or more amino acids of SEQ ID NO:1, a consecutive series of 800 or more amino acids of SEQ ID NO:1, a consecutive series of 1000 or more amino acids of SEQ ID NO:1, a consecutive series of 1500 or more amino acids of SEQ ID NO:1, a consecutive series of 2000 or more amino acids of SEQ ID NO:1, a consecutive series of 2500 or more amino acids of SEQ ID NO:1, a consecutive series of 3000 or more amino acids of SEQ ID NO:1, a consecutive series of 3500 or more amino acids of SEQ ID NO:1, or a consecutive series of 4000 or more amino acids of SEQ ID NO:1. One of ordinary skill in the art would understand how to generate a such a polypeptide using techniques well-known to those of ordinary skill in the art.

In some embodiments of the present invention, the PsrP polypeptide comprises the C-terminus of SEQ ID NO:1. For example, the polypeptide may comprise amino acids 1-50 of SEQ ID NO:1, amino acids 1-100 of SEQ ID NO:1, amino acids 1-150 of SEQ ID NO:1, amino acids 1-200 of SEQ ID NO:1, amino acids 1-250 of SEQ ID NO:1, amino acids 1-300 of SEQ ID NO:1, amino acids 1-350 of SEQ ID NO:1, amino acids 1-400 of SEQ ID NO:1, amino acids 1-450 of SEQ ID NO:1, amino acids 1-500 of SEQ ID NO:1. or amino acids 1-1000 of SEQ ID NO:1.

In other embodiments, the PsrP polypeptide comprises the N-terminus of SEQ ID NO:1. For example, the polypeptide may comprise the N-terminal 50 amino acids of SEQ ID NO:1, the N-terminal 100 amino acids of SEQ ID NO:1, the N-terminal 150 amino acids of SEQ ID NO:1, the N-terminal 200 amino acids of SEQ ID NO:1, the N-terminal 250 amino acids of SEQ ID NO:1, the N-terminal 300 amino acids of SEQ ID NO:1, the N-terminal 350 amino acids of SEQ ID NO:1, the N-terminal 400 amino acids of SEQ ID NO:1, the N-terminal 450 amino acids of SEQ ID NO:1, the N-terminal 500 amino acids of SEQ ID NO:1, or the N-terminal 100 amino acids of SEQ ID NO:1.

In some embodiments, the PsrP polypeptides of the present invention comprise a serine-rich region of SEQ ID NO:1. For example, the PsrP polypeptide may comprise a consecutive series of amino acids of SEQ ID NO:1, wherein at least about 20% of the amino acids are serine residues, wherein at least about 25% of the amino acids are serine residues, wherein at least about 30% of the amino acids are serine residues, a consecutive series of amino acids wherein at least 35% of the amino acids are serine residues, a consecutive series of amino acids wherein at least 40% of the amino acids are serine residues, a consecutive series of amino acids wherein at least 45% of the amino acids are serine residues, or a consecutive series of amino acids wherein at least 50% of the amino acids are serine residues. The consecutive series of amino acids can be of any length, such as a consecutive series of 10, 12, 14, 16, 18, 20, 22, 24, 26, 28, 30, 32, 34, 36, 38, 40, 42, 44, 48, 50, 55, 60, 65, 70, 75, 80, 85, 90, 100 or more amino acids of SEQ ID NO:1. The consecutive series of amino acids may include any amino acid SEQ ID NO:1, such as amino acid 1, 5, 10, 15, 20, 30, 50, 70, 100, 130, 150, 180, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, or 4700 of SEQ ID NO:1.

Some embodiments of the compositions and methods set forth herein concern polypeptides that are equivalent of the PsrP polypeptides set forth above (hereinafter “PsrP polypeptide equivalents”). It is well understood by the skilled artisan that there is a limit to the number of changes that may be made within a defined portion of the molecule and still result in a molecule with an acceptable level of equivalent biological activity, e.g., ability of bind an antibody or to induce an immune response against a bacterium, such as Strep. pneumoniae. “PsrP polypeptide equivalent” is thus defined herein as any PsrP polypeptide in which some, or most, of the amino acids may be substituted so long as the polypeptide retains substantially similar activity in the context of the uses set forth herein.

In some embodiments, the PsrP polypeptide may include an amino acid segment that has a certain percent identity to a consecutive series of polypeptides of SEQ ID NO:1. Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

For example, the amino acid segment may have at least 50% sequence identity, at least 55% sequence identity, at least 60% sequence identity, at least 65% sequence identity, at least 70% sequence identity, and least 75% sequence identity, at lest 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence of SEQ ID NO:1. The consecutive series of polypeptides of SEQ ID NO:1 may be of any length, and may include any sequence of polypeptides of SEQ ID NO:1 as discussed above.

Of course, a plurality of distinct proteins/polypeptides/peptides with different substitutions may easily be made and used in accordance with the invention. Additionally, in the context of the invention, an equivalent can be a PsrP homolog or ortholog polypeptide from any species or organism, including, but not limited to, a human polypeptide. One of ordinary skill in the art will understand that many equivalents would likely exist and can be identified using commonly available techniques.

The present invention may utilize PsrP polypeptides purified from a natural source or from recombinantly-produced material. Those of ordinary skill in the art would know how to produce these polypeptides from recombinantly-produced material. This material may use the common amino acids in naturally synthesized proteins, or one or more modified or unusual amino acids. Generally, “purified” will refer to a composition that has been subjected to fractionation to remove various other proteins, polypeptides, or peptides, and which composition substantially retains its activity. Purification may be substantial, in which the polypeptide is the predominant species, or to homogeneity, which purification level would permit accurate degradative sequencing.

Amino acid sequence mutants of PsrP also are encompassed by the present invention, and are included within the definition of “PsrP polypeptide equivalent.” Amino acid sequence mutants of the polypeptide can be substitutional mutants or insertional mutants. Insertional mutants typically involve the addition of material at a non-terminal point in the peptide. This may include the insertion of a few residues; an immunoreactive epitope; or simply a single residue. The added material may be modified, such as by methylation, acetylation, and the like. Alternatively, additional residues may be added to the N-terminal or C-terminal ends of the peptide.

Amino acid substitutions are generally based on the relative similarity of the amino acid side-chain substituents, or example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

Amino acid substitutions are generally based on the relative similarity of the amino acid side-chain substituents, or example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

In making changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte and Doolittle, 1982, incorporated by reference herein). It is known that certain amino acids may be substituted for other amino acids having a similar hydropathic index or score and still retain a similar biological activity. In making changes based upon the hydropathic index, the substitution of amino acids whose hydropathic indices are within +2 is preferred, those which are within +1 are particularly preferred, and those within +0.5 are even more particularly preferred.

It is understood that an amino acid can be substituted for another having a similar hydrophilicity value and still obtain a biologically equivalent protein. As detailed in U.S. Pat. No. 4,554,101, the following hydrophilicity values have been assigned to amino acid residues: arginine (+3.0); lysine (+3.0); aspartate (+3.0+1); glutamate (+3.0+1); serine (+0.3); asparagine (+0.2); glutamine (+0.2); glycine (0); threonine (−0.4); proline (−0.5+1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within +2 is preferred, those which are within +1 are particularly preferred, and those within +0.5 are even more particularly preferred.

2. PsrP-Encoding Polynucleotides

Various aspects of the present invention require polynucleotides encoding a PsrP polypeptide or PsrP polypeptide equivalent. The polynucleotide may be a nucleic acid segment encoding a PsrP polypeptide as set forth above.

The polynucleotides may be derived from any source known to those of ordinary skill in the art. For example, the polynucleotide may be synthesized using any method known to those of ordinary skill in the art or obtained from natural sources. Nucleic acids may be obtained by chemical synthesis methods or by recombinant methods.

“Nucleic acid” or “polynucleotide” used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference.

Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH.sub.2, NHR, NR.sub.2 or CN, wherein R is C.sub.1-C.sub.6 alkyl, alkenyl or alkynyl and halo is F, Cl, Br or I. Modified nucleotides also include nucleotides conjugated with cholesterol through, e.g., a hydroxyprolinol linkage as described in Krutzfeldt et al. (2005); Soutschek et al. (2004); and U.S. Patent Publication No. 20050107325, which are incorporated herein by reference. Modified nucleotides and nucleic acids may also include locked nucleic acids (LNA), as described in U.S. Patent No. 20020115080, which is incorporated herein by reference. Additional modified nucleotides and nucleic acids are described in U.S. Patent Publication Nos. 20050182005, which is incorporated herein by reference. Modifications of the ribose-phosphate backbone may be done for a variety of reasons, e.g., to increase the stability and half-life of such molecules in physiological environments, to enhance diffusion across cell membranes, or as probes on a biochip.

Mixtures of naturally occurring nucleic acids and analogs may be made; alternatively, mixtures of different nucleic acid analogs, and mixtures of naturally occurring nucleic acids and analogs may be made. It may be advantageous to combine portions of the genomic DNA with cDNA or synthetic sequences to generate specific constructs. For example, where an intron is desired in the ultimate construct, a genomic clone will need to be used. Introns may be derived from other genes. The cDNA or a synthesized polynucleotide may provide more convenient restriction sites for the remaining portion of the construct and, therefore, would be used for the rest of the sequence.

In some embodiments, the polynucleotide as set forth above is operatively coupled to a promoter. “Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

Within certain embodiments expression vectors are employed to express a nucleic acid of interest, such as a miRNA that encodes a PsrP polypeptide as set forth herein. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

There are a number of ways in which expression vectors may introduced into cells. In certain embodiments of the invention, the expression construct comprises a virus or engineered construct derived from a viral genome. One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized. Other examples of vectors include retroviruses, lentiviruses, and so forth.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In a particular example, the oligonucleotide may be administered in combination with a cationic lipid. Examples of cationic lipids include, but are not limited to, lipofectin, DOTMA, DOPE, and DOTAP. The publication of WO/0071096, which is specifically incorporated by reference, describes different formulations, such as a DOTAP:cholesterol or cholesterol derivative formulation that can effectively be used for gene therapy.

### B. ANTIGENS AND VACCINES

Certain embodiments of the present invention involves the use of polypeptides disclosed herein to “immunize” subjects or as “vaccines”. As used herein, “immunization” or “vaccination” means increasing or activating an immune response against an antigen. It does not require elimination or eradication of a condition but rather contemplates the clinically favorable enhancement of an immune response toward an antigen. The vaccine may be a prophylactic vaccine or a therapeutic vaccine. A prophylactic vaccine comprises one or more epitopes associated with a disorder for which the individual may be at risk. Therapeutic vaccines comprise one or more epitopes associated with a particular disorder affecting the individual, such as tumor associated antigens in cancer patients.

As used herein, “vaccine” means an organism or material that contains an antigen in an innocuous form. The vaccine is designed to trigger an immunoprotective response. The vaccine may be recombinant or non-recombinant. When inoculated into a non-immune host, the vaccine will provoke active immunity to the organism or material, but will not cause disease. Vaccines may take the form, for example, of a toxoid, which is defined as a toxin that has been detoxified but that still retains its major immunogenic determinants; or a killed organism, such as typhoid, cholera and poliomyelitis; or attenuated organisms, that are the live, but non-virulent, forms of pathogens, or it may be antigen encoded by such organism, or it may be a live tumor cell or an antigen present on a tumor cell.

“Epitope” refers to an antigenic determinant of a peptide, polypeptide, or protein; an epitope comprises three or more amino acids in a spatial conformation unique to the epitope. Generally, an epitope consists of at least 5 such amino acids and more usually consists of at least 8 to 10 amino acids. Methods of determining spatial conformation of amino acids are known in the art and include, for example, x-ray crystallography and 2-dimensional nuclear magnetic resonance. Antibodies that recognize the same epitope can be identified in a simple immunoassay showing the ability of one antibody to block the binding of another antibody to a target antigen.

Certain embodiments of the present invention pertain to methods of inducing an immune response to an antigen in a subject. The term “antigen” means a substance that is recognized and bound specifically by an antibody or by a T cell antigen receptor. Antigens can include peptides, proteins, glycoproteins, polysaccharides, complex carbohydrates, sugars, gangliosides, lipids and phospholipids; portions thereof and combinations thereof. The antigens can be those found in nature or can be synthetic. Preferably, antigens elicit an antibody response specific for the antigen. Haptens are included within the scope of “antigen.” A hapten is a low molecular weight compound that is not immunogenic by itself but is rendered immunogenic when conjugated with an immunogenic molecule containing antigenic determinants. Small molecules may need to be haptenized in order to be rendered antigenic. Preferably, antigens of the present invention include peptides and polypeptides. In this regard, the immunogenic polypeptides set forth herein include an antigen polypeptide.

An antigen polypeptide is an amino acid sequence that under appropriate conditions results in an immune response in a subject. The immune response may be a an antibody response. For example, the antibody response can be measured as an increase in antibody production, as measured by any number of techniques well-known to those of ordinary skill in the art (e.g., ELISA). The immune response may also be a T cell response, such as increased antigen presentation to T cells, or increased proliferation of T cells.

In some embodiments, the antigen polypeptide is administered with the intent of inducing an immune response. Depending on the intended mode of administration, the compounds of the present invention can be in various pharmaceutical compositions. The compositions will include a unit dose of the selected polypeptide in combination with a pharmaceutically acceptable carrier and, in addition, can include other medicinal agents, pharmaceutical agents, carriers, adjuvants, diluents, and excipients. “Pharmaceutically acceptable” means a material that is not biologically or otherwise undesirable, i.e., the material can be administered to an individual along with the fusion protein or other composition without causing any undesirable biological effects or interacting in a deleterious manner with any of the other components of the pharmaceutical composition in which it is contained.

Preparation of vaccines and immunizing agents is generally well understood in the art, as exemplified by U.S. Pat. Nos. 4,608,251; 4,601,903; 4,599,231; 4,599,230; 4,596,792; and 4,578,770, all incorporated herein by reference. Typically, such vaccines are prepared as injectables either as liquid solutions or suspensions; solid forms suitable for solution in, or suspension in, liquid prior to injection may also be prepared. The preparation may also be emulsified. In addition, if desired, the vaccine may contain minor amounts of auxiliary substances such as wetting or emulsifying agents, pH buffering agents, or adjuvants which enhance the effectiveness of the vaccines.

Examples of physiologically acceptable carriers include saline solutions such as normal saline, Ringer's solution, PBS (phosphate-buffered saline), and generally mixtures of various salts including potassium and phosphate salts with or without sugar additives such as glucose. The active immunogenic ingredient is often mixed with excipients which are pharmaceutically acceptable and compatible with the active ingredient. Suitable excipients are, for example, water, saline, dextrose, glycerol, ethanol, or the like, and combinations thereof. Nontoxic auxiliary substances, such as wetting agents, buffers, or emulsifiers may also be added to the composition. Oral formulations include such normally employed excipients as, for example, pharmaceutical grades of mannitol, lactose, starch, magnesium stearate, sodium saccharine, cellulose, magnesium carbonate, and the like. In one embodiment of the invention, adjuvants are not required for immunization.

Parenteral administration, if used, is generally characterized by injection. Sterile injectables can be prepared in conventional forms, either as liquid solutions or suspensions, solid forms suitable for solution or suspension in liquid prior to injection, or as emulsions.

The vaccine compositions set forth herein may comprise an adjuvant and/or a carrier. Adjuvants are any substance whose admixture into the vaccine composition increases or otherwise modifies the immune response to an antigen.

Adjuvants could for example be selected from the group consisting of: A1K(SO₄)₂, AlNa(SO₄)₂, AlNH(SO₄)₄, silica, alum, AI(OH)₃, Ca₃(PO₄)₂, kaolin, carbon, aluminum hydroxide, muramyl dipeptides, N-acetyl-muramyl-L-threonyl-D-isoglutamine (thr-DMP), N-acetyl-nornuramyl-L-alanyl-D-isoglutamine (CGP 11687, also referred to as nor-MDP), N-acetylmuramyul-L-alanyl-D-isoglutaminyl-L-alanine-2-(1′2′-dipalmitoyl-s-n-glycero-3-hydroxphosphoryloxy)-ethylamine (CGP 19835A, also referred to as MTP-PE), RIBI (MPL+TDM+CWS) in a 2% squalene/Tween-80® emulsion, lipopolysaccharides and its various derivatives, including lipid A, Freund's Complete Adjuvant (FCA), Freund's Incomplete Adjuvants, Merck Adjuvant 65, polynucleotides (for example, poly IC and poly AU acids), wax D from Mycobacterium, tuberculosis, substances found in Corynebacterium parvum, Bordetella pertussis, and members of the genus Brucella, liposomes or other lipid emulsions, Titermax, ISCOMS, Quil A, ALUN (see U.S. Pat. No. 5,554,372), Lipid A derivatives, choleratoxin derivatives, HSP derivatives, LPS derivatives, synthetic peptide matrixes or GMDP, Interleukin 1, Interleukin 2, Montanide ISA-51 and QS-21.

Other agents which stimulate the immune response can also be administered to the subject. For example, other cytokines are also useful in vaccination protocols as a result of their lymphocyte regulatory properties. Many other cytokines useful for such purposes will be known to one of ordinary skill in the art, including interleukin-12 (IL-12) which has been shown to enhance the protective effects of vaccines, GM-CSF and IL-18. Thus cytokines can be administered in conjunction with antigens and adjuvants to increase the immune response to the antigens.

A vaccine composition according to the present invention may comprise more than one different adjuvant. Furthermore, the invention encompasses a therapeutic composition further comprising any adjuvant substance including any of the above or combinations thereof. It is also contemplated that ML-IAP, or one or more fragments thereof, and the adjuvant can be administered separately in any appropriate sequence.

In certain embodiments, the vaccine composition includes a carrier. The carrier may be any suitable carrier known to the person skilled in the art, for example a protein or an antigen presenting cell. Examples include serum proteins such as transferrin, bovine serum albumin, human serum albumin, thyroglobulin or ovalbumin, immunoglobulins, or hormones, such as insulin or palmitic acid. For immunization of humans, the carrier must be a physiologically acceptable carrier acceptable to humans and safe. However, tetanus toxoid and/or diptheria toxoid are suitable carriers in one embodiment of the invention. Alternatively, the carrier may be dextrans for example sepharose.

The timing of administration of the vaccine and the number of doses required for immunization can be determined from standard vaccine administration protocols. Typically a vaccine composition will be administered in two doses. The first dose will be administered at the elected date and a second dose will follow at one month from the first dose. A third dose may be administered if necessary, and desired time intervals for delivery of multiple doses of a particular antigen containing HCH2 polymer can be determined by one of ordinary skill in the art employing no more than routine experimentation. The antigen containing HCH2 polymer may be given as a single dose.

For each recipient, the total vaccine amount necessary can be deduced from protocols for immunization with other vaccines. The exact amount of antigen-HCH2 polymer required will vary from subject to subject, depending on the species, age, weight and general condition of the subject, the particular fusion protein used, its mode of administration, and the like. Generally, dosage will approximate that which is typical for the administration of other vaccines, and will preferably be in the range of about 10 ng/kg to 1 mg/kg.

Methods for the preparation of mixtures or emulsions of polypeptides disclosed herein and adjuvant are well known to those of skill in the art of vaccination (see, e.g. Plotkin and Orenstein, 2004).

Immunizations against toxins and viral infection can be tested using in vitro assays and standard animal models. For example a mouse can be immunized with a viral antigen polypeptide expressed as a fusion protein with HCH2 polymers and delivered by the methods detailed herein. After the appropriate period of time to allow immunity to develop against the antigen, for example two weeks, a blood sample is tested to determine the level of antibodies, termed the antibody titer, using ELISA. In some instances the mouse is immunized and, after the appropriate period of time, challenged with the virus to determine if protective immunity against the virus has been achieved. Using these techniques the proper combination of antigen, adjuvant, and other vaccine components can be optimized to boost the immune response. Testing in humans can be contemplated after efficacy is demonstrated in animal models. Methods for immunization, including formulation of a vaccine composition and selection of doses, route of administration and the schedule of administration (e.g. primary and one or more booster doses), are well known in the art (e.g. see Vaccines: From concept to clinic, 1999).

Generally accepted animal models can be used for testing of immunization against cancer using a tumor and cancer antigen polypeptides. For example, cancer cells (human or murine) can be introduced into a mouse to create a tumor, and one or more cancer associated antigens can be delivered by the methods described herein. The effect on the cancer cells (e.g., reduction of tumor size) can be assessed as a measure of the effectiveness of the immunization. Of course, immunization can include one or more adjuvants and/or cytokines to boost the immune response. The tests also can be performed in humans, where the end point is to test for the presence of enhanced levels of circulating cytotoxic T lymphocytes against cells bearing the antigen, to test for levels of circulating antibodies against the antigen, to test for the presence of cells expressing the antigen and so forth.

In some embodiments, the vaccine composition includes antigen presenting cells. The antigen presenting cell can be a dendritic cells (DC). DC may be cultivated ex vivo or derived in culture from peripheral blood progenitor cells (PBPC) and peripheral blood stem cells (PBSC). The dendritic cells may be prepared and used in therapeutic procedures according to any suitable protocol known to those of ordinary skill in the art. It will be appreciated by the person skilled in the art that the protocol may be adopted to use with patients with different HLA types and different diseases. Incubation of cultured dendritic cells with HCH2 polymers of the invention is envisaged as a means of loading dendritic cells with antigen for subsequent transfer into hosts.

For any of the ex vivo methods of the invention, peripheral blood progenitor cells (PBPC) and peripheral blood stem cells (PBSC) are collected using apheresis procedures known in the art. Briefly, PBPC and PBSC are collected using conventional devices, for example, a Haemonetics®. Model V50 apheresis device (Haemonetics, Braintree, Mass.). Four-hour collections are performed typically no more than five times weekly until, for example, approximately 6.5.times.10.sup.8 mononuclear cells (MNC)/kg patient are collected. The cells are suspended in standard media and then centrifuged to remove red blood cells and neutrophils. Cells located at the interface between the two phases (also known in the art as the buffy coat) are withdrawn and resuspended in HBSS. The suspended cells are predominantly mononuclear and a substantial portion of the cell mixture are early stem cells. The stem cells obtained in this manner can be frozen, then stored in the vapor phase of liquid nitrogen. Ten percent dimethylsulfoxide can be used as a cryoprotectant. After all collections from the donor have been made, the stem cells are thawed and pooled. Aliquots containing stem cells, growth medium, such as McCoy's 5A medium, 0.3% agar, and expansion factors (e.g. GM-CSF, IL-3, IL-4, flt3-ligand), are cultured and expanded at 37 degrees Celsius in 5% CO₂ in fully humidified air for 14 days.

### C. IMMUNOLOGICAL REAGENTS

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody fragment” is used to refer to any antibody-like molecule that does not fall within the definition of antibody but which includes an antigen-binding domain. Examples include Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, 1988; incorporated herein by reference).

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

However, “humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide, peptide or domain, be it a wild-type or mutant composition. The immunizing composition is administered in a manner effective to stimulate antibody producing cells.

### D. PHARMACEUTICAL PREPARATIONS

Pharmaceutical preparations of PsrP polypeptides, or antibodies and/or antibody fragments for administration to a subject are contemplated by the present invention.

1. Formulations

Any type of pharmaceutical preparation is contemplated by the current invention. One of skill in art would be familiar with the wide range of types of pharmaceutical preparations that are available, and would be familiar with skills needed to generate these pharmaceutical preparations.

In certain embodiments of the present invention, the pharmaceutical preparation will be an aqueous composition. Aqueous compositions of the present invention comprise an effective amount an PsrP polypeptide, and the like, dissolved or dispersed in a pharmaceutically acceptable carrier or aqueous medium. Aqueous compositions of gene therapy vectors expressing any of the foregoing are also contemplated. The phrases “pharmaceutical preparation suitable for delivery” or “pharmacologically effective” of “pharmaceutically acceptable” refer to molecular entities and compositions that do not produce an adverse, allergic or other untoward reaction when administered to an animal, or a human, as appropriate.

As used herein, “pharmaceutical preparation” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. For human administration, preparations should meet sterility, pyrogenicity, general safety and purity standards as required by FDA Office of Biologics standards.

The biological material should be extensively dialyzed to remove undesired small molecular weight molecules and/or lyophilized for more ready formulation into a desired vehicle, where appropriate. The active compounds will then generally be formulated for administration by any known route, such as parenteral administration. The preparation of an aqueous composition containing an active agent of the invention disclosed herein as a component or active ingredient will be known to those of skill in the art in light of the present disclosure.

An agent or substance of the present invention can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. A person of ordinary skill in the art would be familiar with techniques for generation of salt forms. The carrier can also be a solvent or dispersion medium containing, for example, water, ethanol, polyol (for example, glycerol, propylene glycol, and liquid polyethylene glycol, and the like), suitable mixtures thereof, and vegetable oils.

The present invention contemplates PsrP polypeptides that will be in pharmaceutical preparations that are sterile solutions for intravascular injection or for application by any other route. A person of ordinary skill in the art would be familiar with techniques for generating sterile solutions for injection or application by any other route. Sterile injectable solutions are prepared by incorporating the active compounds in the required amount in the appropriate solvent with various of the other ingredients familiar to a person of skill in the art.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure.

The active agents disclosed herein may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds formulated for parenteral administration, such as intravenous or intramuscular injection, other pharmaceutically acceptable forms include, e.g., tablets or other solids for oral administration; liposomal formulations; time release capsules; and any other form currently used, including cremes. One may also use nasal solutions or sprays, aerosols or inhalants in the present invention.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. A person of ordinary skill in the art would be familiar with well-known techniques for preparation of oral formulations. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The use of liposomes and/or nanoparticles is also contemplated for the introduction of the modulator of cell death or gene therapy vectors into host cells. The formation and use of liposomes is generally known to those of skill in the art.

Administration of the pharmaceutical compositions of the present invention may be by any method known to those of ordinary skill in the art. For example, administration may be topical, local, regional, systemic, by aerosol, by spray, intravenous, intradermal, intraarterial, intramuscular, intrathecal, intratracheal, subcutaneous, or intraperitoneal. Oral compositions are also contemplated by the present invention.

2. Dosage

An effective amount of the therapeutic or preventive agent is determined based on the intended goal, for example treatment or prevention of a bacterial infection in a subject. The quantity to be administered, both according to number of treatments and dose, depends on the subject to be treated, the state of the subject and the protection desired. Precise amounts of the therapeutic composition also depend on the judgment of the practitioner and are peculiar to each individual.

For example, a dose of the therapeutic agent may be about 0.0001 milligrams to about 1.0 milligrams, or about 0.001 milligrams to about 0.1 milligrams, or about 0.1 milligrams to about 1.0 milligrams, or even about 10 milligrams per dose or so. Multiple doses can also be administered. In some embodiments, a dose is at least about 0.0001 milligrams. In further embodiments, a dose is at least about 0.001 milligrams. In still further embodiments, a dose is at least 0.01 milligrams. In still further embodiments, a dose is at least about 0.1 milligrams. In more particular embodiments, a dose may be at least 1.0 milligrams. In even more particular embodiments, a dose may be at least 10 milligrams. In further embodiments, a dose is at least 100 milligrams or higher.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The dose can be repeated as needed as determined by those of ordinary skill in the art. Thus, in some embodiments of the methods set forth herein, a single dose is contemplated. In other embodiments, two or more doses are contemplated. Where more than one dose is administered to a subject, the time interval between doses can be any time interval as determined by those of ordinary skill in the art. For example, the time interval between doses may be about 1 hour to about 2 hours, about 2 hours to about 6 hours, about 6 hours to about 10 hours, about 10 hours to about 24 hours, about 1 day to about 2 days, about 1 week to about 2 weeks, or longer, or any time interval derivable within any of these recited ranges.

3. Secondary Treatment

Certain embodiments of the claimed invention provide for a method of treating or preventing an infection in a subject. Some of the methods set forth herein involve administering to the subject one or more secondary forms of therapy directed to the treatment or prevention of a bacterial infection. Examples of such therapies include other vaccines directed to prevention or treatment of infection due to Streptococcus pneumoniae, or antibiotics. Any such therapy known to those of ordinary skill in the art is contemplated as a secondary form of therapy.

### E. TREATMENT AND PREVENTION OF DISEASE

“Treatment” and “treating” as used herein refer to administration or application of a therapeutic agent to a subject or performance of a procedure or modality on a subject for the purpose of obtaining a therapeutic benefit of a disease or health-related condition. For example, treatment of pneumonia may involve administration of a therapeutic agent for the reduction in symptoms of pneumonia, such as reduction in cough or improvement in respiratory function.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition.

The disease to be treated or prevented may be any bacterial infection. For example, the bacterial infection may be a Staphylococcus species, an E. coli, a streptococcus species, a chlamydia, salmonella, vibrio cholerae, Treponema pallidum, Neisseria gonorrhoeae, a borrelia species, a mycobacterium species, a Yersinia species, or a bacillus species. In particular embodiments, the bacterial is a streptococcus. Non-limiting examples of streptococcus species include S. parasanguinis, S. peroris, S. pneumoniae, S. pyogenes, S. ratti, S. salivarius, S. salivarius ssp. Thermophilus, S. sanguinis, or S. viridans. In specific embodiments, the strep. species is S. pneumoniae.

Non-limiting examples of diseases contemplated for treatment include, but are not limited to, diseases of the respiratory tract, diseases of the gastronintestinal tract, diseases of the skin, disease of the central nervous system, diseases of the heart. Non-limiting more particular examples include pneumonia, bronchitis, endocarditis, sepsis, abscesses, meningitis, toxic shock syndrome, erysipelas, scarlet fever, rheumatic fever, Streptococcal pharyngitis, enterocolitis, gastritis, necrotizing enteritis, and so forth. In a particular embodiment, the disease is pneumonia due to S. pneumoniae.

### F. KITS

The technology herein includes kits. For example, a kit may include, for example, one or more components such as a sealed containing including a PsrP polypeptide or a nucleic acid encoding a PsrP polypeptide. The kits may optionally include a reagent, an instruction sheet, and other elements useful to practice the technology described herein. These physical elements can be arranged in any way suitable for carrying out the invention.

Kits can include further buffers, enzymes, labeling compounds, and the like. Any of the compositions described herein may be comprised in a kit.

The kit may include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a single vial. The kits of the present invention also will typically include a means for containing the nucleic acids or polypeptides set forth herein, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, such as a sterile aqueous solution.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale.

### G. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Antibodies Against PsrP, a Novel Streptococcus pneumoniae Adhesion, Block Adhesion and Protect Mice Against Pneumococcal Challenge

### Materials and Methods

Bacterial strains, media, DNA and RNA isolation. TIGR4 (Tettelin et al., 2001), and its mutant derivatives T4ÄpsrP and T4ÄpsrP-secY2A2 were grown in Todd-Hewitt (TH) broth (Difco Laboratories, Detroit, Mich.) or on blood agar plates with 1 μg/mL erythromycin, as needed, at 37° C. in 5% CO₂. Plasmids were maintained in Escherichia coli strain DH5α grown on Luria-Bertani (LB) media with 100 μg/mL ampicillin. DNA and RNA were isolated using standard protocols (Sambrook et al., 1989).

Creation of T4ΔpsrP-secY2A2. T4ΔpsrP was previously created by insertion duplication mutagenesis (Obert et al., 2006). To test T4ΔpsrP for polar effects, Northern blot analysis was performed using radio-labelled probes for SP1773, psrP (SP1772) and glyA (SP1771) (Sambrook et al., 1989). T4ΔpsrP-secY2A2 was created by allelic exchange (Horton, 1995). The erythromycin resistance cassette ermB was PCR amplified from plasmid pTCV-lac (Poyart and Trieu-Cuot, 1997) and cloned into the TA cloning vector pCR2.1 (Invitrogen, Carlsbad Calif.). DNA fragments representing sequences at the 5′ and 3′ extreme of psrP-secY2A2 (gam: TIGR annotation SP1757; psrP: TIGR annotation SP1772) were amplified from genomic DNA and cloned upstream and downstream of the ermB cassette in pCR2.1: gtfB F: 5′-NNNNNACTAGTGGAGATAATCAGTCTGCTTG (SEQ ID NO:2); gtfB R: 5′-NNNNNAAGCTTGCGCGCTCATAAGTCGCC (SEQ ID NO:3); psrP F: NNNNNTCTAGAGGCAAGTACATCTGCATCTG (SEQ ID NO:4); psrP R: NNNNNGCGGCCGCGATAGAATATCCAGGACG (SEQ ID NO:5) (N for the mixed nucleotide bases). The resulting mutagenic construct (˜3 kb), containing the ermB cassette and the two flanking DNA fragments, was PCR amplified and used to transform TIGR4 (Bricker and Camilli, 1999). Deletion of the 35-kb locus was confirmed by successful amplification of a PCR product using primers that flanked the deleted genes, sequencing the PCR product, and failure to amplify genes previously located within psrP-secY2A2 (data not shown). Polar effects were tested for by Northern blot analysis.

Characterization of the mutants. Exponential phase TIGR4, T4ΔpsrP and T466 psrP-secY2A2 were used to create frozen glycerol stocks. Glass tubes containing 10 mL of TH media were inoculated with these stocks to obtain an initial concentration of 10⁵ colony forming units (cfu)/mL. Microbial growth was determined by measuring the optical density of the cultures at OD₆₂₀ on an hourly basis for 8 hours. The ability to undergo autolysis was determined by addition of sodium deoxycholate (final concentration 0.1%) to the cultures and monitoring a decline in optical density. Competence was tested for using published methods (Bricker and Camilli, 1999). Presence of capsule was confirmed by Quelling reaction using serotype-4 specific antiserum (Statens Serum Institute, Copenhagen, Denmark). Levels of capsule were determined using the Stains-all Assay (Sigma) for detecting polysaccharides (Hammerschmidt et al., 2005). Briefly, polysaccharide values from T4R, an unencapsulated derivative of TIGR4 (Gosink et al., 2000), was subtracted from those of test bacteria to determine the amount of capsule present. Each experiment was performed in triplicate.

Mice experiments. Female BALB/cJ mice, 4-5 weeks old, were obtained from The Jackson Laboratory (Bar Harbor, Me.). For all experiments mice were anesthetized with isoflurane prior to challenge. Exponential phase cultures of S. pneumoniae were centrifuged, washed, and suspended in sterile phosphate buffered saline (PBS). For intranasal challenge, each mouse was instilled with 10⁷ cfu in 25 μL of PBS into the left nostril. Two days post-challenge, mice were sacrificed and bacterial titers in the nasal lavage determined by serial dilution of nasal elute and plating of aliquots. The number of bacteria in the lungs was assessed per gram of homogenized tissue. For intratracheal challenge, 10⁵ cfu in 100 μL of PBS was placed in the throat of mice hung upright by their incisors. Aspiration of the bacteria was induced by gently pulling the tongue outward and covering the nostrils. Two days later, mice bacterial titers in the lungs and blood were determined. For intraperitoneal challenge (i.p.), mice were injected with 10⁵ cfu in 100 μL of PBS. One day after challenge blood was collected by heart puncture. For passive immunization experiments, one day before intranasal challenge mice were administered i.p. 250 μL of antiserum against recombinant (r)PsrP_{SRR1-BR}, rSP0925, or naïve serum from the same rabbit (see below). Bacterial titers in the nasopharynx, lungs and blood were determined at day two. For each challenge model and bacteria strain tested, two replicate experiments were performed each with 4-6 mice for a minimum of 8 mice sampled. All experiments were performed in compliance with an approved Institutional Animal Care and Use Committee protocol. Statistical analysis was performed using a 1-Way ANOVA.

Recombinant PsrPSRR1-BR and development of antiserum. The coding region for PsrP domains SRR1-BR was PCR amplified from chromosomal DNA. The 5′ primer started at the nucleotide encoding the first codon of the SRR1 domain: SRR1 F: 5′-NNNNNGAATTCTCAGCGAGTTCAACTAGTTTG (SEQ ID NO:6). The 3′ primer started at the nucleotide encoding the last amino acid of the BR: BR R: 5′-NNNNNAAGCTTTTAACTAGCACTTACTG (SEQ ID NO:7). Primers were designed to allow directional cloning of the PCR product into the expression vector pRSET-A (Invitrogen) which added an N-terminal hexahistidine sequence to the expressed protein. Transformed E. coli strain BL21 (DE3) (Novagen, Madison, Wis.) was grown aerobically at 37° C. in LB broth. Production of rPsrP_{SRR1-BR} was induced by addition of isopropyl-β-D-thiogalactoside to a concentration of 1 mM when the culture had reached an OD₆₀₀=0.6. Six hours later cells were harvested and the His-tagged protein was purified under denaturing conditions using a Ni-nitrilotriacetic acid column with a QIAexpress Ni-NTA Fast Start Kit (Qiagen, Valencia, Calif.). Protein in the eluted fractions was made visible by coomassie blue staining of SDS-PAGE gels loaded with fraction samples. The fraction containing a single band corresponding to the predicted size of rPsrP_{SRR1-BR} (45-kDa) was dialyzed extensively against PBS and stored at −20° C. Purified rPsrP_{SRR1-BR} was sent to Invitrogen Custom Antibody & Peptide Services (Carlsbad, Calif.) for creation of rabbit antiserum using their standard protocol. Specificity of the antiserum was confirmed by ELISA and Western blot. Cloning, purification of, and production of antiserum against rSP0925, an integral membrane protein, were done in the same manner described for rPsrP_{SRR1-BR}. Primers used were: SP0925 F: 5′-NNNNNGAATTCATGAAAAAACGAGC (SEQ ID NO:8); SP0925 R: 5′-NNNNNAAGCTTACTTCCTGAAAATAGGAGC (SEQ ID NO:9)).

Adhesion assays. Detroit 562 cells (human nasopharyngeal epithelial cells; ATCC CCL-138), A549 cells (human alveolar type II pneumocytes; ATCC CRL-185), HBMEC cells (human brain microvasculature endothelial cells; ScienCell, Carlsbad, Calif.); LA-4 cells (murine bronchial epithelial cells; ATCC CCL-196) and RBCEC₆ cells (rat capillary endothelial cell line; provided by Elaine Tuomanen, Memphis, Tenn.) were grown to 100% confluence on 24-well plates (˜10⁶ cells/well). Prior to their use, cells were washed with cell line-specific tissue culture media to remove serum. Cells were exposed to media containing 10⁷ cfu/mL of bacteria and incubated for 1 hour at 37° C. in 5% CO₂. Non-adherent bacteria were removed by washing the cells 3 times with PBS and the number of adherent bacteria was determined by lysis of the monolayer with 0.1% Triton X-100 and plating of the lysate. For fluorescent bead assays, rPsrP_{SRR1-BR} or bovine serum albumin (BSA) was adsorbed to 10⁸ Fluoresbrite® YG Microspheres 1.00 μm (Polysciences Inc, Warrington, Pa.) following the manufacturer's instruction. The amount of protein on each bead was calculated by subtracting the amount of protein left in the supernatant following adsorption from the amount used initially (300 μg/mL) and dividing by the number of beads adsorbed. Bead assays were performed in 24-well plates with 10⁷ beads/mL and 10⁶ A549 cells. After 30 minute incubation, cells were washed, and attached beads visualized and counted using an inverted fluorescent microscope. For competitive inhibition binding assays, A549 cells were incubated with increasing concentrations of rPsrP_{SRR1-BR} for 30 minutes, the cells washed, and the bacteria added to the cells. Likewise, for antibody inhibition assays, TIGR4 was incubated in tissue culture media containing antiserum for 30 minutes prior to its wash and use. All adhesion experiments were performed in triplicate with a minimum of 3 wells per experiment. Statistical analysis was performed using a 1-Way ANOVA.

### Results

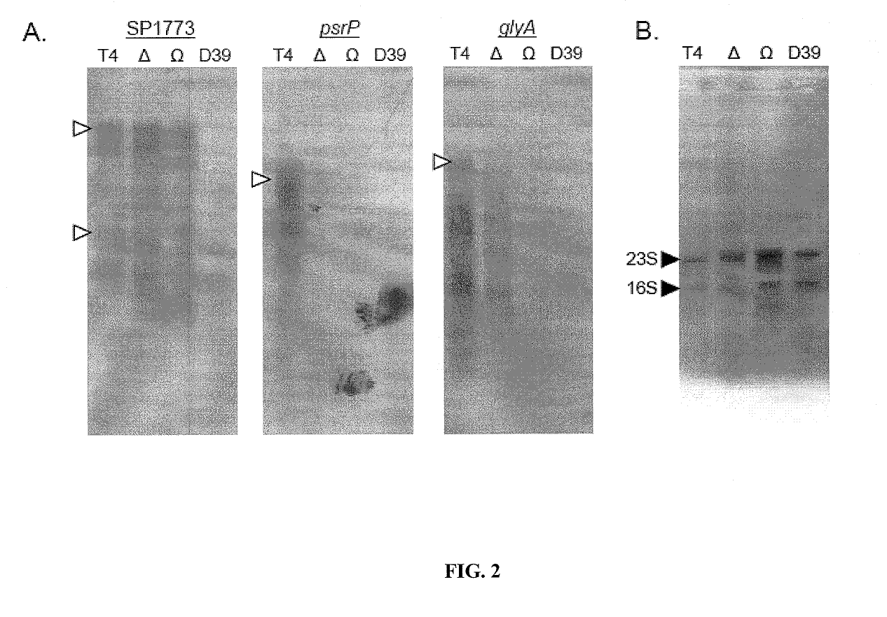

Deletion of psrP-secY2A2 does not affect in vitro fitness. Northern blot analysis of psrP transcription and that of the genes immediately upstream (SP1773) and downstream (glyA) of psrP, suggests that psrP has its own promoter and that its mutation does not have polar effects. FIG. 2 demonstrates that expression of SP1773 and glyA are unchanged in T4ΔpsrP versus TIGR4, whereas expression of psrP is abrogated. Transformation of TIGR4 with the gtfB:ermR:psrP mutagenic PCR product resulted in creation of mutant that was deficient from gtfB to psrP; 16 genes spanning >36,000 bp. Two small overlapping genes, asp4 and asp5, were left in the chromosome without their promoter to ensure that polar effects did not occur. Of note, genes downstream of asp5 are transcribed in the opposite orientation and should be unaffected. Northern blots demonstrated that no upstream effects (i.e. altered SP1773 expression) occurred in T4ΔpsrP-secY2A2 (FIG. 2). Assessment of the mutants in vitro determined that deletion of psrP or psrP-secY2A2 did not negatively effect bacterial growth in TH media, colony morphology, the presence and amount of capsular polysaccharide, autolysis, or the ability to undergo transformation (data not shown).

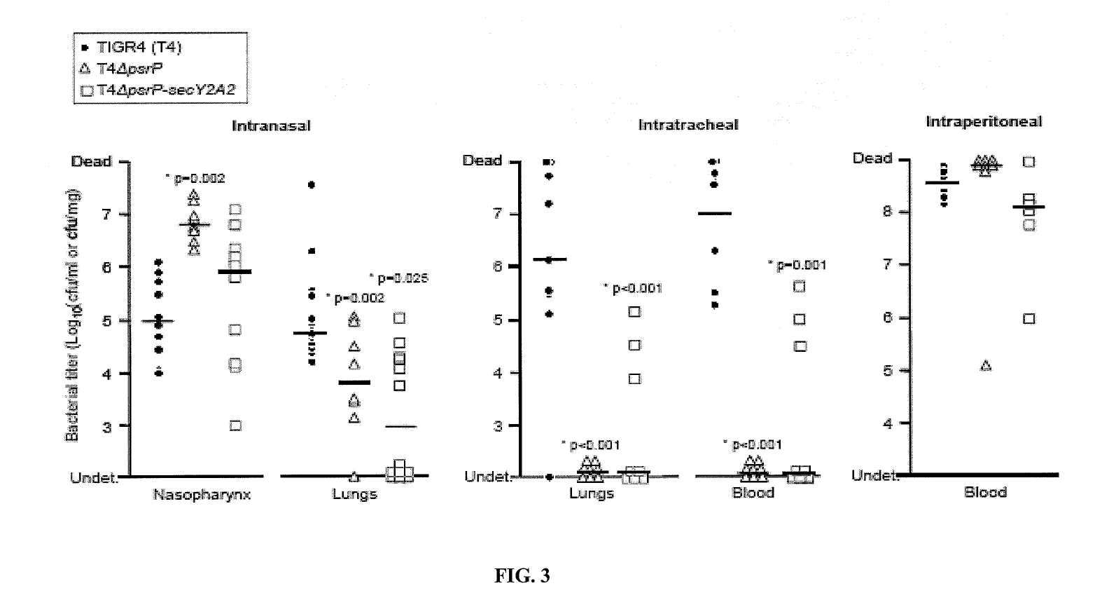

PsrP is required in the lungs. To determine the anatomical site-specific contribution of PsrP and to ascertain if its accessory glycosylases and SecY2A2 transport machinery contributed to virulence, the inventors infected mice with TIGR4, T4ΔpsrP, and T4ΔpsrP-secY2A2 via intranasal, intratracheal, and intraperitoneal challenge routes (FIG. 3). Two-days post challenge, mice infected intranasally with T4ΔpsrP and T4ΔpsrP-secY2A2 had a greater or equivalent number of bacteria in the nasopharynx than mice infected with TIGR4. Importantly, the same mice had median bacterial titers in the lungs 10 and 100-fold less than mice infected with TIGR4, respectively. Intratracheal challenge of mice exacerbated the requirement for psrP or psrP-secY2A2 in vivo. Mice infected with TIGR4 had a median bacterial titer greater than 10⁶ cfu/g or mL in the lungs and blood, whereas mice infected with T4ΔpsrP or T4ΔpsrP-secY2A2 had median bacterial titers lower than the detectable threshold (<10³ cfu/g or mL). Intraperitoneal challenge of mice with the mutants failed to discern any differences in the number of bacteria in the blood or in mortality versus wild type. In all instances mice died within 36 hours (data not shown). Thus psrP-secY2A2 was not required for nasopharyngeal colonization or for replication in the blood following i.p. injection. However, psrP and psrP-secY2A2 were required in the lungs, and the mutants were attenuated in their ability to progress to the blood and cause high-grade bacteremia.

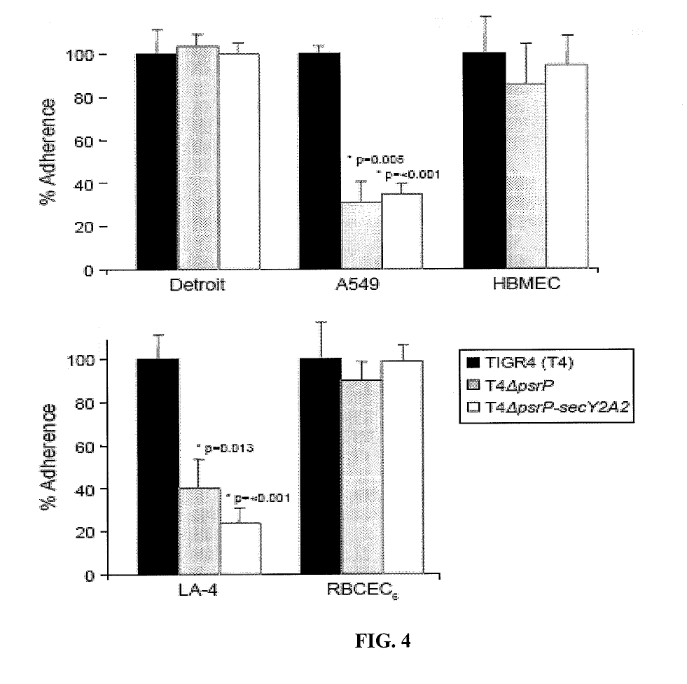

psrP-secY2A2 contributes towards adhesion to lungs cells but not to other cell types. Because SRRPs are adhesins, the inventors tested the ability of TIGR4 and the mutants to attach to a panel of tissue culture cell lines. Adhesion assays determined that the PsrP-deficient mutants were attenuated in their ability to bind to human alveolar epithelial cells (A549) and mouse bronchial epithelial cells (LA-4). T4ΔpsrP and T4ΔpsrP-secY2A2 attached to A549 cells at 34% and 38% the level of TIGR4, respectively, and attached to LA-4 cells at 40% and 21% the level of TIGR4, respectively (FIG. 4). In contrast, mutant bacteria adhered normally to human pharyngeal epithelial cells (Detroit), and human and rodent brain microvasculature endothelial cells (HBMEC and RBCEC₆); findings that corroborate the observed lung-specific role for PsrP in vivo.

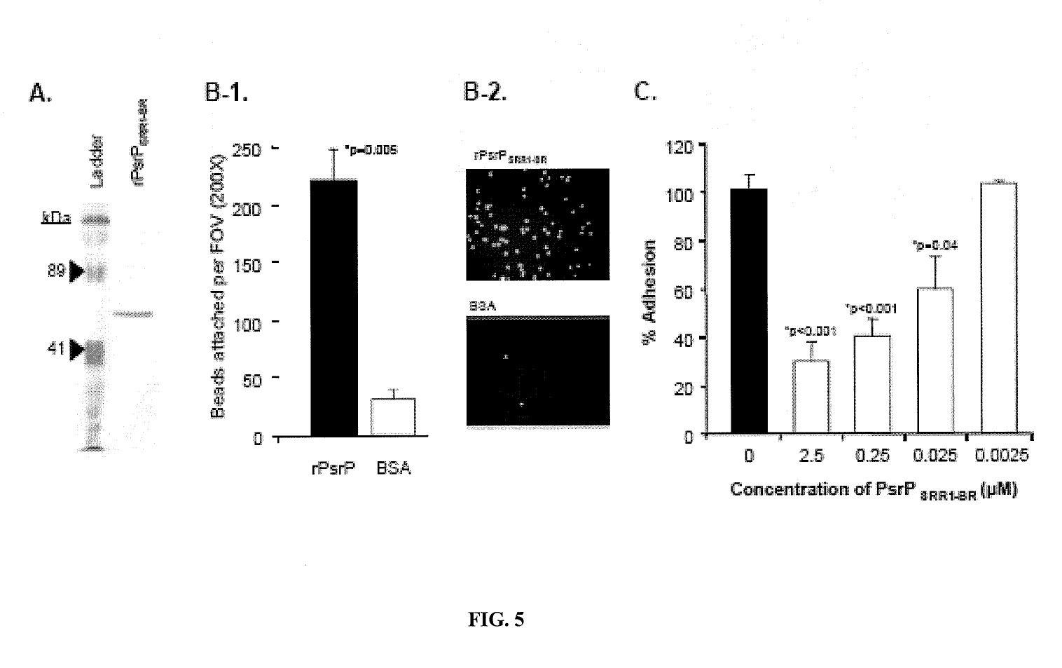

rPsrP_{SRR1-BR} binds to A549 cells and competitively inhibits S. pneumoniae adhesion. SRRPs mediate adhesion through their BR. To determine if PsrP did the same, the inventors coated fluorescent latex microspheres with recombinant protein composed of the SRR1 and BR domains (rPsrP_{SRR1-BR}) (FIG. 5A) or BSA and compared their ability to bind to A549 cells (10-20 pg protein/bead; based on ELISA equivalent to 100× amount of PsrP on individual TIGR4 [data not shown]). rPsrP_{SRR1-BR} coated microspheres adhered to cells at levels 6.4-fold greater than beads coated with BSA (FIG. 5B). Pre-incubation of A549 cells with rPsrP_{SRR1-BR} inhibited bacteria adhesion in a dose-dependent manner (FIG. 5C). A549 cells treated with 2.5 μM, 0.25 μM, 0.025 μM, and 0.0025 μM rPsrP bound 30±8%, 40±6%, and 60±13%, and 103±2%, the number of wild type bacteria that adhered to untreated cells, respectively. These findings suggest that the amino-terminus of PsrP, contributes towards bacterial attachment to A549 cells.

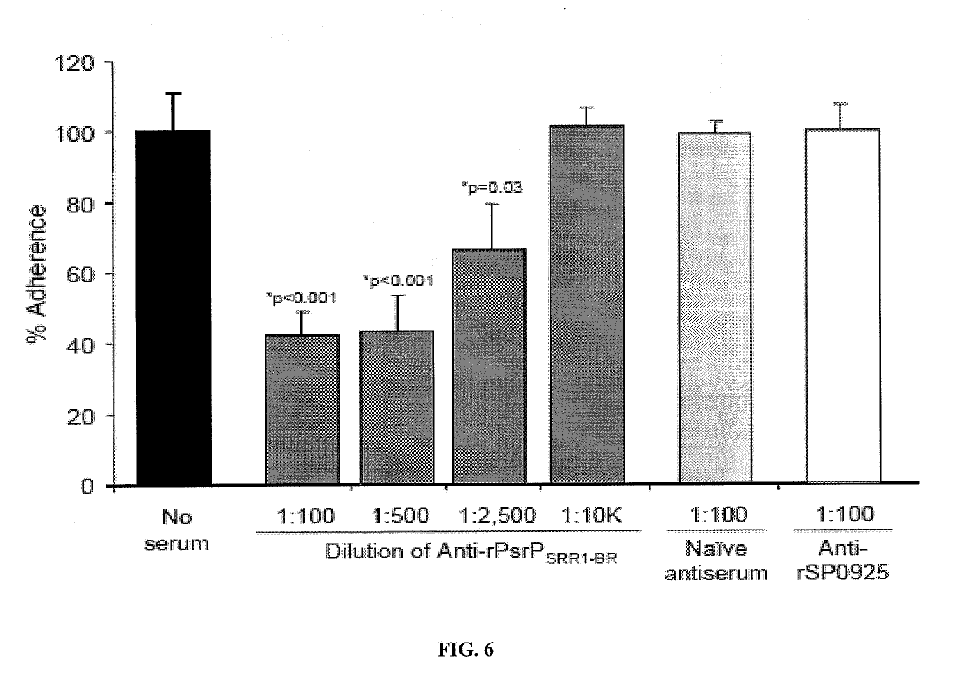

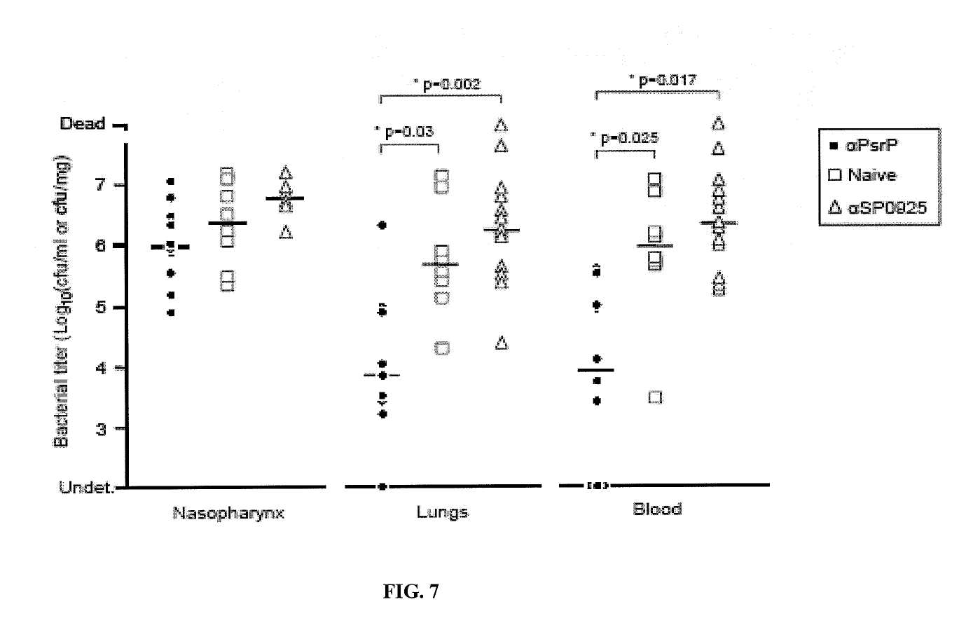

Antiserum against rPsrPSRR1-BR inhibits adhesion and protects against challenge. To further test if SRR1-BR mediated PsrP adhesion, and to determine if antibodies against these domains inhibit bacteria adhesion, the inventors performed a series of experiments with antiserum to rPsrP_{SRR1-BR}, naïve antiserum, and antiserum to an unrelated S. pneumoniae integral membrane protein (SP0925). Pre-incubation of TIGR4 with rPsrP_{SRR1-BR} antiserum inhibited adhesion in a dose-dependent manner (FIG. 6). Decreased adhesion was observed at serum dilutions of 1:100, 1:500, and 1:2,500 but not at 1:10,000. Naïve serum and rSP0925 antiserum had no effect on the ability of TIGR4 to adhere (1:100 dilutions). Passive immunization of mice with rPsrP_{SRR1-BR} antiserum conferred protection against challenge with TIGR4. Mice who received rPsrPBR antiserum had approximately 100-fold fewer bacteria in the lungs and blood than those who received naïve or SP0925 antiserum. Passive immunization had no effect on bacterial titers in the nasopharynx (FIG. 7).

In summary, the inventors have determined that PsrP is a S. pneumoniae adhesin that it is required for adherence in vitro and persistence in the lower respiratory tract. The studies have demonstrated that PsrP adhesion is mediated by the amino terminus of the protein, moreover, that antibodies against the SRR1 and BR domain inhibit S. pneumoniae adhesion and protect mice against pneumococcal challenge. Future studies will focus on identifying the ligand for PsrP in the lungs and determining if active vaccination confers protective immunity.

### Example 2

### Distribution of PsrP Among Clinical Isolates

To further examine if PsrP is broadly distributed among clinically significant serotypes. The clonal complex of each of the 6 sequenced genomes which we have been determined to contain psrP-secY2A2 were identified. Invasive pneumococcal disease (IPD) is primarily caused by a small number of invasive clones within each serotype (Sandgren et al., 2004). Some of these invasive clones exist in multiple serotypes. Because the incidence of invasive disease associated with each serotype/clonal complex in the United States has recently been published (Beall et al. 2001), by identifying the clonal complex (using MLST) the incidence of invasive disease associated with strains that putatively carry psrP-secY2A2 (Supplemental Data Table 1) were determined.

SUPPLEMENTAL DATA TABLE 1
Clonal complexes associated with psrP-secY2A2 and associated incidence of IPD

| | | | No. invasive isolates in the clonal complex/total number of | | | | | |
|---|---|---|---|---|---|---|---|---|
| | | Serotypes with clinical isolates | isolates for each serotype tested from 1999-2002** | | | | | |
| Isolate^{(serotype)} | Clonal complex* | belonging to the clonal complex | <5 years | | ≧5 years | | total | |
| INV104B¹ | 227 | 1 | 13/14 | (92.9%) | 43/49 | (87.8%) | 59/63 | (88.9%) |
| TIGR4⁴ | 205 | 4 | 14/106 | (13.2)% | 11/34 | (32.4%) | 25/141 | (17.7%) |
| SP6-BS73^{6A} | 460 | 6A | 27/108 | (25.0%) | 3/51 | (5.9%) | 30/159 | (18.9%) |
| | | 10A | 7/7 | (100%) | 4/7 | (57.1%) | 11/14 | (78.6%) |
| | | 23F | 2/37 | (5.4%) | 0/18 | (0.0%) | 2/55 | (3.6%) |
| INV-200¹⁴ | ST14 | 14 | 255/299 | (85.3%) | 31/39 | (79.5%) | 286/338 | (84.6%) |
| CDC-3059-06^{19A} | 199 | 15BC | 53/61 | (86.9%) | 8/8 | (100.0%) | 61/69 | (88.4%) |
| | | 19A | 58/87 | (66.7%) | 88/119 | (73.9%) | 146/206 | (70.9%) |
| | | 19F | 11/159 | (6.9%) | 1/21 | (4.7%) | 12/180 | (6.7%) |
| Spain-23F^{23F} | 81 | 19A | 2/58 | (3.4%) | 2/45 | (4.4%) | 4/108 | (3.9%) |
| | | 19F | 16/159 | (10.1%) | 3/21 | (14.3%) | 19/180 | (10.6%) |
| | | 23F | 16/101 | (15.8%) | 9/18 | (50%) | 25/119 | (21.0%) |

*Clonal complex determined by MLST using sequence data available through GenBank.
**Beall, B., M. C. McEllistrem, R. E. Gertz, Jr., S. Wedel, D. J. Boxrud, A. L. Gonzalez, et al.,
Pre- and postvaccination clonal compositions of invasive pneumococcal serotypes for isolates collected in the United States in 1999, 2001, and 2002. J Clin Microbiol, 2006. 44(3): p. 999-1017

In summary, it was determined that invasive clones that carry psrP-secY2A2 are responsible for up to:

88.9% of invasive disease that is a result of infection with serotype 1*
88.4% of invasive disease that is a result of infection with serotype 15BC*
84.6% of invasive disease that is a result of infection with serotype 14
78.6% of invasive disease that is a result of infection with serotype 10A*
74.8% of invasive disease that is a result of infection with serotype 19A*
24.6% of invasive disease that is a result of infection with serotype 23F
18.9% of invasive disease that is a result of infection with serotype 6A*
17.7% of invasive disease that is a result of infection with serotype 4
17.3% of invasive disease that is a result of infection with serotype 19F
*Not present in the 7-valent conjugate vaccine

Thus, clones that carry psrP-secY2A2 are widely distributed, cause a significant amount of morbidity and mortality, and are present in non-vaccine serotypes. These findings highlight the need to better understand PsrP-mediated adhesion and its contribution to development of invasive disease.

All of the methods disclosed and claimed herein can be made and executed without undue experimentation in light of the present disclosure. While the methods of this invention have been described in terms of preferred embodiments, it will be apparent to those of skill in the art that variations may be applied to the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,196,265
U.S. Pat. No. 4,578,770
U.S. Pat. No. 4,596,792
U.S. Pat. No. 4,599,230
U.S. Pat. No. 4,599,231
U.S. Pat. No. 4,601,903
U.S. Pat. No. 4,608,251
U.S. Pat. No. 5,554,372
ACIP, In: Recommendations of the Advisory Committee on Immunization Practices, MMWR Recomm. Rep., 49:1-35, 2000.
Antibodies: A Laboratory Manual, Cold Spring Harbor Laboratory, 1988.
Beal et al., J. Clin. Microbiol., 44(3):999-1017, 2006.
Bensing and Sullam, Mol. Microbiol., 44:1081-1094, 2002.
Bensing et al., J. bacteriol., 186:638-645, 2004.
Bensing et al., Mol. Microbiol., 58:1468-1481, 2005.
Bricker and Camilli, FEMS Microbiol. Lett., 172:131-135, 1999.
Chen et al., Mol. Microbiol., 53843-856, 2004.
Froeliger and Fives-Taylor, Infect. Immun., 69:2512-2519, 2001.
Gosink et al., Infect. Immun., 68:5690-5695, 2000.
Hammerschmidt et al., Infect. Immun., 73:4653-4667, 2005.
Handley et al., Oral Microbiol. Immunol., 20:131-140, 2005.
Horton, Mol. Biotechnol., 3:93-99, 1995.
Obert et al., Infect. Immun., 74:4766-4777, 2006.
Plotkin and Orenstein, In: Vaccines, 4^{th} Ed., 2004.
Poyart and Trieu-Cuot, FEMS Microbiol. Lett., 156:193-198, 1997.
Sambrook et al., Molecular Cloning: A Laboratory Manual, Cold Spring Harbor, Cold Spring Harbor Press, 1989.
Sandgren et al., J. Infect. Dis., 189:785-796, 2004.
Siboo et al., Infect. Immun., 73:2273-2280, 2005.
Takahashi et al., Infect. Immun., 70:1209-1218, 2002.
Takahashi et al., Infect. Immun., 74:740-743, 2006.
Takamatsu et al., Infect. Immun., 74:1933-1940, 2006.
Takamatsu et al., J. Bacteriol., 187:3878-3883, 2005.
Takamatsu et al., J. Bacteriol., 196:7100-7111, 2004.
Takamatsu et al., Mol. Microbiol., 52:189-203, 2004.
Takamatsu et al., Mol. Microbiol., 58:380-392, 2005.
Tettelin et al., Science, 293:498-506, 2001.
Vaccines: From concept to clinic, Paoletti and McInnes (Eds.), CRC Press, 1999.
WHO Position Paper, Pneumococcal vaccines, Weekly Epidemoil. Rec., 74:177-193, 1999.
Wu et al., Mol. Microbiol., 28:487-500, 1998.

1-56. (canceled)

57. A method of treating or preventing a bacterial infection in a subject comprising administering to the subject an effective amount of a pharmaceutical composition comprising:
a) a PsrP polypeptide, a nucleic acid encoding a PsrP polypeptide, or an antibody or antibody fragment that binds to a PsrP polypeptide; and
b) a pharmaceutically acceptable carrier,
wherein the bacterial infection is treated or prevented.

58. The method of claim 57, wherein the subject is a human.

59. The method of claim 57, wherein the subject has pneumonia.

60. The method of claim 57, wherein the bacterial infection is a Streptococcus pneumoniae infection.

61. The method of claim 57, wherein the pharmaceutical composition comprises a PsrP polypeptide.

62. The method of claim 61, wherein the PsrP polypeptide comprises 50 to 4000 consecutive amino acids of SEQ ID NO:1.

63. The method of claim 61, wherein the PsrP polypeptide comprises SEQ ID NO:1.

64. The method of claim 61, wherein the PsrP polypeptide comprises a region that has at least 95% sequence identity to a consecutive series of at least 100 amino acids of SEQ ID NO:1.

65. The method of claim 61, wherein the PsrP polypeptide comprises at least 100 consecutive amino acids of SEQ ID NO:1, wherein at least about 30% of the amino acid residues in the at least 100 consecutive amino acids of SEQ ID NO:1 are serine residues.

66. The method of claim 61, wherein the pharmaceutical composition comprises a PsrP polypeptide expressed on the surface of an antigen-presenting cell.

67. The method of claim 57, wherein the pharmaceutical composition comprises a nucleic acid encoding a PsrP polypeptide.

68. The method of claim 57, wherein the pharmaceutical composition comprises an antibody or antibody fragment.

69. The method of claim 68, wherein the antibody or antibody fragment binds to a basic domain of PsrP or a serine-rich repeat region of PsrP.

70. A pharmaceutical composition, comprising:
(a) a PsrP polypeptide, a nucleic acid encoding a PsrP polypeptide, or an antibody or antibody fragment that binds to a PsrP polypeptide; and
(b) a pharmaceutically acceptable carrier;
wherein the composition induces a humoral or cell-mediated immune response when administered to a subject.

71. The pharmaceutical composition of claim 70, wherein the composition comprises a PsrP polypeptide.

72. The pharmaceutical composition of claim 70, further comprising an antigen-presenting cell comprising the PsrP polypeptide.

73. The pharmaceutical composition of claim 70, further comprising an adjuvant.

74. The pharmaceutical composition of claim 71, wherein the PsrP polypeptide comprises 50 to 4000 consecutive amino acids of SEQ ID NO:1.

75. The pharmaceutical composition of claim 71, wherein the PsrP polypeptide comprises SEQ ID NO:1.

76. A kit comprising a sealed vial comprising a pharmaceutical composition comprising:
a) a PsrP polypeptide, a nucleic acid encoding a PsrP polypeptide, an antibody that binds to a PsrP polypeptide, or an antibody fragment that binds to a PsrP polypeptide; and
b) a pharmaceutically acceptable carrier.

77. The kit of claim 76, further comprising a syringe.
